(19) United States
(12) Patent Application Publication
(10) Pub. No.: US 2010/0170867 A1
(43) Pub. Date: Jul. 8, 2010

(54) PACKAGING CONTAINER AND APPARATUS AND METHOD FOR MANUFACTURING SAME

(75) Inventor: Atsushi Hayakawa, Shinjuku-ku (JP)

Correspondence Address: SUGHRUE MION, PLLC, 2100 PENNSYLVANIA AVENUE, N.W., SUITE 800, WASHINGTON, DC, 20037, US

(73) Assignee: DAI NIPPON PRINTING CO., LTD, Shinjuku-ku, Tokyo (JP)

(21) Appl. No.: 12/676,100

(22) PCT Filed: Aug. 26, 2008
(86) PCT No.: PCT/JP2008/065172

§ 371 Date: Mar. 2, 2010

(30) Foreign Application Priority Data
Sep. 3, 2007 (JP) 2007-228104
May 14, 2008 (JP) 2008-127010

(51) Int. Cl. B65B 55/02; A61L 2/18; A61L 2/20

(52) U.S. Cl. 215/379; 422/28; 422/304; 422/27; 422/295

(57) ABSTRACT

There is provided a packaging container which reserves an acidic beverage having a relatively high acidity and allows spore to remain properly without corrupting and the acidic beverage can be aseptically filled and reserved at a normal temperature with low cost without utilizing a container having high heat-proof property and expensive manufacturing equipment. An interior of a container 2 is sterilized by a sterilizing agent “b” and a heated water “c” so as to allow bacterial spore to be alive but not to allow bacteria vegetative cell, mold and yeast to be alive, and a sterilized content “a” having an acidity of an extent of suppressing germination of the bacteria spore fills the container at a normal temperature or low temperature, and then the container is sealed by a lid 3, thereby providing a packaging container 1.

### FIELD OF THE INVENTION

The present invention relates to a packaging container which is a container such as bottle to be filled with inner content such as beverage or drink in commercially aseptic manner, and also relates to apparatus and method for manufacturing the packaging container.

Herein, it is to be noted that the packaging container of the present invention is a package with a cap, in shape of container such as bottle which is filled up with an inner content such as beverage or drink, and thereafter, sealed with a lid, and in this meaning, the present invention will be referred to as “packaging container” and a term “container” means a package body before being filled by the inner content.

### BACKGROUND ART

(1) According to Food Sanitation Law, although it is not required, for acidic beverage (pH<4) to which a predetermined carbon dioxide pressure is applied, to be sterilized, in a case where such acidic beverage includes composition or component of plant or animal, it is required for the beverage to be sterilized in spite of the presence or absence of the carbon dioxide pressure. Because of this Law, in a case of a beverage containing carbon dioxide of less the pH 4.0 including composition or component of plant or animal (for example, lactic carbonate drink, fruit juice carbonate drink, or fruit colored carbonate drink), it is required to be heated for 10 minutes at temperature of 65° C.

Such sterilization is performed by filling a heat-resisting and pressure-resisting bottle with, for example, acidic beverage, and sealing the bottle by applying a cap, and then pouring shower of heated water of a temperature of about 65 to 75° C. onto the heat-resisting and pressure-resisting bottle, thus sterilizing the bottle, the cap and the inner content.

(2) Furthermore, according to Food Sanitation Law, in a case of a beverage having pH 4.0 to pH 4.6 (for example, vegetable-type beverage such as tomato juice, vegetable juice or like), it is required to be heated for 30 minutes at temperature of 30° C.

For such sterilization, a so-called hot-pack method is generally adapted as sterilization method. In such hot-pack method, beverage is heated, for example, to a temperature of 90 to 140° C. so as to sterilize the beverage itself, which then fills the bottle at a temperature of 85 to 95° C. to thereby sterilize the inner surface of the bottle. The bottle is then sealed with a cap, thereafter, the bottle is turned upside-down so as to sterilize the inner surface of the cap, and then, the bottle is cooled stepwise by a pasteurizer (an apparatus for pasteurization) so as to produce a packaging container. According to such hot-pack method, the heat-resisting bottle and the cap as well as the beverage can be sterilized.

In a case where the bottle is made of PET (polyethylene telephthalate), if the bottle is sterilized by heated water of a temperature more than 85° C., the bottle may be deformed by heat. In order to prevent such deformation, the inner surface of the bottle is cleaned by intermittently jetting hot water of a temperature of 65 to 85° C., and thereafter, the bottle is filled with acidic beverage at a normal temperature and is then sealed. Such method has been also provided (for example, refer to Patent Document 6).

(3) Furthermore, according to Food Sanitation Law, in a case of a beverage having pH of more than pH 4.6 (for example, tea-type beverage or drink including red tea such as milk tea, green tea, barley tea or mixed tea), it is required to sterilize the beverage by a method of completely achieving an effect of killing virus, which may be grew in the beverage.

An aseptic method will be adopted for manufacturing packaging container for such beverage under aseptic condition. This aseptic method is a method in which a bottle is preliminarily heated during traveling in an aseptic atmosphere (environment), the bottle is sterilized by hydrogen peroxide mist as sterilizing agent, washed and cleaned and filled with sterilized beverage at a room temperature. Thereafter, the bottle is sealed with a cap, thus manufacturing the aseptic packaging container (for example, refer to Patent Document 1).

In addition, in the hot-pack method mentioned above, beforehand the filling operation, a path, as a product line, from a beverage blending tank to a filler for applying the beverage into the bottle, is sterilized by a sterilizing method similar to that for sterilizing the beverage itself.

This sterilizing method for the product line will be performed, for example, by circulating heated water of a temperature of 85° C., for example, in a conduit or duct of the product line for about 30 minutes.

After the circulation of the heated water, the conduit is not cooled, and the beverage heated to the predetermined temperature fills the bottle passing through the product line, the inside of which is maintained in the sterilized state by the heated beverage.

In the aseptic method mentioned above, prior to the beverage filling working, the path, as the product line, from the beverage blending tank to the filler by which the bottle is filled with the beverage is subjected to the sterilization process or treatment by the sterilizing method substantially corresponding to the sterilization of the beverage mentioned hereinbefore.

This sterilization process to the product line may be performed by combined use of the hydrogen peroxide and steam, for example, (refer to Patent Document 3, for example), in general, such sterilization process is performed by flowing steam of a temperature 120 to 130° C. into the conduit for about 20 to 30 minutes. Thereafter, aseptic air is fed into the conduit to be thereby cooled, and when the temperature is lowered to the normal temperature (about 20 to 40° C., which may be different from inner contents filling the bottles), the filling process starts.

Furthermore, in both the hot-pack method and the aseptic method mentioned above, before the filling working starting, the inside of the aseptic chamber surrounding the aseptic packaging apparatus may be preliminarily sterilized (for example, refer to Patent Documents 2, 4 and 5).

The path from the bottle sterilization to the sealing of the bottle with a cap through the filling of the beverage is covered by the aseptic chamber, and also, the inside of the aseptic chamber itself is subjected to the sterilization process or treatment beforehand the filling working by the sterilizing method substantially the same sterilization for the beverage and bottles as mentioned hereinbefore.

As conventional sterilizing method for the aseptic chamber, the following methods are provided, such as: a method in which peracetic acid spray, aseptic water introduction, hot air introduction hydrogen peroxide spray and hot air introduction are subsequently performed (for example, refer to Patent Document 2); a method in which sterilization by hydrogen peroxide group medical agent and heated water cleaning are performed in this order (for example, refer to Patent Document 4); and a method in which a material which is prepared by mixing a sterilizing agent such as hydrogen peroxide or peracetic acid (acetyl hydrogen peroxide) with air is blasted into the aseptic chamber before the starting of the filling working to the actual filling working time (for example, refer to Patent Document 5).

Patent Document 1: Japanese Patent Laid-open Publication No. 2001-39414

Patent Document 2: Japanese Patent Publication No. 3315918

Patent Document 3: Japanese Patent Laid-open Publication No. SHO 57-93061

Patent Document 4: Japanese Patent Laid-open Publication No. 2008-168930

Patent Document 5: Japanese Patent Laid-open Publication No. HEI 9-328113

Patent Document 6: Japanese Patent Publication No. 2844983

### Means for Solving the Problem

According to the sterilizing methods (1) and (2) mentioned above, although bacteria vegetative cells of molds and yeasts in virus may be sterilized, spores of the bacteria is alive (remains alive) without being sterilized or killed, and almost all bacteria spores remain alive in its bacteriostatic state without germinating in acidic beverage (drink) having relatively high degree of acidity (for example, vegetable juice having pH of less than 4.6, tomato juice, lemon juice, orange juice, lactic carbonated drink (soda), functional drink, carbon-contained lemon juice, grape juice, fruit juice, or like) without some acidophilic bacteria or germs, and because of this reason, the beverage can be preserved without being corrupted.

However, when such sterilizing method (1) as mentioned above in which the heated water shower is jetted on the bottle, or the hot-pack type sterilizing method (2) also mentioned above are adopted, it is necessary for the bottle to endow heat-resisting property. That is, in order to prevent the bottle mouth portion from being deformed by heat and to prevent the inner beverage from leaking outward, it is necessary to heat the mouth portion of the bottle so as to crystallize the mouth portion if the bottle is made of PET. Furthermore, in a case when hot drink fills the bottle and a lid is screwed to fasten the mouth portion and then the bottle is kept for some time in this state, the bottle may be shrunk by reduction of inner pressure. In order to absorb such shrinkage (shrinking amount of the bottle), it will be required to provide a reduced pressure absorption panel to a side surface or a bottom surface of the bottle. Various workings or countermeasures against such heat change may cause cost-increasing for manufacturing the packaging container.

If heated water of the temperature not causing any deformation to the PET bottle is utilized, the above mentioned defects or drawbacks may be overcome, but in such case, there is a fear in temperature management of the heated water because if such management is not sufficiently performed, the inside of the bottle may be inadequately sterilized. For example, it is hard for molds having high heat resisting property to be sufficiently sterilized with the heated water of such temperature, and the molds may survive. Furthermore, an in-bottle sterilizing step, an inner content filling step, a capping step and like step are performed under an aseptic environment covered by the aseptic chamber. However, if the bottle sterilizing treatment is performed only by the heated water, there may cause a case where survived virus adheres to the bottle or virus floating in air invades into the aseptic environment, and in such case, the survived virus may invade into the bottle together with the inner content, which may contaminate the interior of the packaging container body.

According to the aseptic method (3) mentioned hereinbefore, since the heat-resisting property is not required for the bottle, the bottle can be manufactured with reduced or cheep cost. This aseptic method, however, is a sterilizing method of killing all the virus including bacteria vegetative cells, molds and yeasts in virus, as well as spores of the bacteria, so that this sterilizing method involves many complicated sterilizing processes, and in addition, a lot of utility such as sterilizing agent, heated water, hot-air and the like are required. Furthermore, since it is required to perform the sterilization treatment to the filling apparatus beforehand the beverage filling process and interiors of chambers surrounding the filling apparatus to sterilize even spores of bacteria, there require sterilizing agents, heated water, and complicated processes and apparatus and the like, and long time is needed for the sterilization. Accordingly, this aseptic method requires excessive facilities or equipments and processes, and hence, is not suitable for acidic beverages having certainly high acidity and allowing spores to remain.

Furthermore, according to the sterilizing method (2) mentioned above, although the bacteria vegetative cells, molds and yeasts in virus in the product line can be sterilized, the spores of the bacteria remain without being sterilized, and the spores of almost all bacteria keep alive in its bacteriostatic state in the product line without germinating in acidic beverage (drink) having relatively high degree of acidity (for example, vegetable juice having pH of less than 4.6, tomato juice, lemon juice, orange juice, lactic carbonated drink (soda), functional drink, carbon-contained lemon juice, grape juice, fruit juice, or like) without some acidophilic bacteria or germs.

However, in order to performing the filling of the inner content while maintaining the sterilized state allowing only the spores to remain, it is necessary to feed the inner content such as beverage in the heated state into the product line. Because of this reason, the sterilization method (2) mentioned above for filling the inner content such as lactic product, which is not suitable to for being applied with the heating process, cannot be adopted.

According to the sterilizing method of the product line corresponding to the aseptic method (3) also mentioned above, after heating the inside of the conduit to about 130° C., it is cooled by aseptic air to normal temperature, so that the sterilization of conduit requires long time such as 1 to 2 hours, and because of this reason, operating time of the aseptic filling machine is lowered, providing a problem.

Furthermore, the sterilization process or treatment in the aseptic chamber before the filling working in a conventional technology has been performed by a method like the aseptic method (3) mentioned above. As mentioned above, the aseptic method is a method of sterilizing all the virus including not only the bacteria vegetative cells, molds and yeasts but also spores of bacteria, and accordingly, this method requires a lot of utilities such as sterilizing agents, heated water, hot-air and the like, and also requires long time for sterilization. Thus, the sterilization process in the aseptic chamber by the aseptic method requires excessive facilities, equipments and processes, and hence, is not suitable for the acidic beverage having certainly high acidity and allowing spores to remain.

Accordingly, the present invention aims to provide means capable of suitably reserving acidic beverage having certainly high acidity and allowing spore to remain without being corroded and filling acidic beverage in a container in aseptic state with low cost without using expensive manufacturing facilities or like and then preserving the same.

Moreover, the present invention aims to provide means capable of sterilizing a product line easily for a short time using for a filling operation in an aseptic condition.

Furthermore, the present invention aims to provide means capable of sterilizing easily for a short time an environment interior of an aseptic chamber in which a filling operation is performed in the aseptic condition.

### Means for Solving the Problem

In order to achieve the above aims, the present invention adopts the following structures.

Further, it is to be noted that for the purpose of easy understanding of the present invention, reference numerals are added in parentheses on the drawings, but the present invention is not limited to embodiments described in the drawings.

That is, the invention according to claim 1 is a packaging container (1, 28) wherein an interior of a container (2) is sterilized by a sterilizing agent (b) and a heated water (c) so as to allow bacterial spore to be alive but not to allow bacteria vegetative cell, mold and yeast to be alive, an inner content (a), which has been subjected to a sterilization treatment, having an acidity suppressing germination of the bacteria spore fills the container (2) at a normal or low temperature, and the container (2) is sealed by a lid (3).

Herein, as the sterilizing agent, for example, hydrogen peroxide or acetyl hydrogen peroxide group sterilizing agent may be used. In the use of the acetyl hydrogen peroxide group sterilizing agent, the interior of the container can be sterilized by rinsing the interior of the container by the acetyl hydrogen peroxide liquid or spraying the same.

As recited in claim 2, in the packaging container (1, 28) recited in claim 1, the sterilizing agent is hydrogen peroxide, and the interior of the container is sterilized by blowing mist or gas (b) of the hydrogen peroxide into the container (2).

As recited in claim 3, in the packaging container (1, 28) recited in claim 2, the mist (b) of the hydrogen peroxide to be fed into the container (2) is of an amount of 5 to 50 μL/container (L: litter).

As recited in claim 4, in the packaging container (1, 28) recited in claim 2, the gas (b) of the hydrogen peroxide to be fed into the container (2) has a gas density of 1 to 5 mg/L to sterilize the interior of the container.

As recited in claim 5, in the packaging container (1, 28) recited in claim 1, the heated water (c) has a temperature of 65 to 85° C. and is fed at a feeding rate of 5 to 15 L/min.

As recited in claim 6, in the packaging container (1, 28) recited in claim 1, the inner content (a) has an acidity of less than pH 4.6.

As recited in claim 7, in the packaging container (1, 28) recited in claim 1, the inner content (a) fills the container at a normal temperature of 3 to 40° C.

As recited in claim 8, in the packaging container (1, 28) recited in claim 1, the inner content is a liquid beverage (a).

As recited in claim 9, in the packaging container (1, 28) recited in claim 1, the container (2) is made of PET (polyethylene telephthalate) material or polyethylene material.

As recited in claim 10, in the packaging container (1, 28) recited in claim 1, the container is a bottle (2).

The invention according to claim 11 is a method of manufacturing a packaging container, comprising the steps of: sterilizing an interior of a container (2) by a sterilizing agent (b) and a heated water (c) so as to allow bacterial spore to be alive but not to allow bacteria vegetative cell, mold and yeast to be alive; filling the container with an inner content (a), which has been subjected to a sterilization treatment, having an acidity suppressing germination of the bacteria spore at a normal or low temperature; and sealing the container (2) by a lid (3).

As recited in claim 12, in the manufacturing method of the packaging container (1, 28) recited in claim 11, the interior of the container (2) is sterilized by blowing mist or gas (b) of a hydrogen peroxide as sterilizing agent into the container (2).

As recited in claim 13, in the manufacturing method of the packaging container (1, 28) recited in claim 11, the mist (b) of the hydrogen peroxide to be fed into the container (2) is of an amount of 5 to 50 μL/container.

As recited in claim 14, in the manufacturing method of the packaging container (1, 28) recited in claim 11, the gas (b) of the hydrogen peroxide to be fed into the container (2) has a gas density of 1 to 5 mg/L.

As recited in claim 15, in the manufacturing method of the packaging container (1, 28) recited in claim 11, the heated water (c) has a temperature of 65 to 85° C. and is fed at a feeding rate of 5 to 15 L/min.

As recited in claim 16, in the manufacturing method of the packaging container (1, 28) recited in claim 11, the inner content (a) has an acidity of less than pH 4.6.

As recited in claim 17, in the manufacturing method of the packaging container (1, 28) recited in claim 11, the inner content (a) fills the container at a normal temperature of 3 to 40° C.

As recited in claim 18, in the manufacturing method of the packaging container (1, 28) recited in claim 11, the inner content (a) is a liquid beverage.

As recited in claim 19, in the manufacturing method of the packaging container (1, 28) recited in claim 11, the container (2) is made of PET (polyethylene telephthalate) material or polyethylene material.

As recited in claim 20, in the manufacturing method of the packaging container (1, 28) recited in claim 11, the container is a bottle (2).

As recited in claim 21, in the manufacturing method of the packaging container (1, 28) recited in claim 11, the bottle (2) is subjected to a blow-molding process from a preform (10) just before the sterilization treatment of the interior of the bottle (2).

As recited in claim 22, in the manufacturing method of the packaging container (1, 28) recited in claim 11, an exterior of the container (2) is sterilized by the sterilizing agent (b) and the interior of the container (2) is sterilized by the heated water (c) in a state in which the sterilizing agent (b) adheres to an outer surface of the container (2).

The invention according to claim 23 is an apparatus for manufacturing a packaging container (1, 28) comprising: a conveying unit that conveys a container (2) along a predetermined conveying root; a first sterilizing unit (5) that is disposed along the conveying root for performing the sterilization of an interior of the container (2) by a sterilizing agent (b) so as to allow bacterial spore to be alive but not to allow bacteria vegetative cell, mold and yeast to be alive; a second sterilizing unit (6) that performs the sterilization of an interior of the container (2) by a heated water (c) so as to allow bacterial spore to be alive but not to allow bacteria vegetative cell, mold and yeast to be alive; a content filling unit (7) that fills the container (2) with an inner content (a), which has been subjected to a sterilization treatment, having an acidity suppressing germination of the bacteria spore at a normal or low temperature; and a sealing unit (8) that seals the container (2) by a lid (3), wherein the respective units are arranged in the described order, and a portion between the first sterilizing unit (5) to the sealing unit (8) is covered by aseptic chambers (23, 24, 26, 27).

In this apparatus, as the sterilizing agent, the hydrogen peroxide or acetyl hydrogen peroxide group sterilizing agent may be used. In the use of the acetyl hydrogen peroxide group sterilizing agent, the interior of the container can be sterilized by rinsing the interior of the container by the acetyl hydrogen peroxide liquid or spraying the same.

As recited in claim 24, in the apparatus for manufacturing a packaging container (1, 28) recited in claim 23, the sterilizing unit is composed of a nozzle (5) for blowing mist or gas (b) of hydrogen peroxide as the sterilizing agent into the container (2), and the nozzle has a front end facing a mouth portion (2a) of the container (2).

As recited in claim 25, in the apparatus for manufacturing a packaging container (1, 28) recited in claim 23, the container is a bottle (2) and a blow-molding unit (9) that molds the bottle from a preform (10) is disposed just before the sterilizing unit.

As recited in claim 26, in the method of manufacturing a packaging container (1, 28) recited in claim 11, with respect to a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive, the interior of the product line is positively pressurized, thereafter, the interior of the product line is cooled to a normal temperature by passing an aseptic water or the content, and the content is fed to the container through the thus cooled product line.

As recited in claim 27, in the method of manufacturing a packaging container (1, 28) recited in claim 26, the heated water has a temperature of 80 to 140° C. and passes through the product line for 1 to 30 minutes.

As recited in claim 28, in the apparatus for manufacturing a packaging container (1, 28) recited in claim 23, the apparatus further comprising: a heating and sterilizing unit that sterilizes and positively pressurizes an interior of a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive; and a cooling unit that cools the interior of the product line to a normal temperature by passing an aseptic water or content through the product line after the sterilization by the heating and sterilizing unit.

As recited in claim 29, in the apparatus for manufacturing a packaging container (1, 28) recited in claim 28, the heated water has a temperature of 80 to 140° C. and passes through the product line for 1 to 30 minutes.

As recited in claim 30, in the apparatus for manufacturing a packaging container (1, 28) recited in claim 28, the inner content has an acidity of less than pH 4.6.

As recited in claim 31, in the method of manufacturing a packaging container (1, 28) recited in claim 11, steps of sterilizing the interior of the container (2), filling the container with the content (a), and sealing the container with the lid (3) are performed inside the aseptic chambers (23, 24, 26, 27), the interiors of the aseptic chambers being preliminarily sterilized by the sterilizing agent and the heated water so as to allow bacterial spore to be alive but not to allow bacteria vegetative cell, mold and yeast to be alive.

As recited in claim 32, in the apparatus for manufacturing a packaging container (1, 28) recited in claim 23, the first sterilizing unit (5), the second sterilizing unit (6), the content filling unit (7) and the sealing unit (8) are covered by the aseptic chambers (23, 24, 26, 27), in which nozzles (78, 29) are disposed so as to inject the sterilizing agent and the heated water successively, and by successively injecting the sterilizing agent and the heated water through the nozzles, the interior of the aseptic chambers (23, 24, 26, 27) are preliminarily sterilized so as to allow bacterial spore to be alive but not to allow bacteria vegetative cell, mold and yeast to be alive.

### EFFECTS OF THE INVENTION

According to the invention of claim 1, there is provided a packaging container (1, 28) wherein an interior of a container (2) is sterilized by a sterilizing agent (b) and a heated water (c) so as to allow bacterial spore to be alive but not to allow bacteria vegetative cell, mold and yeast to be alive, an inner content (a), which has been subjected to a sterilization treatment, having an acidity suppressing germination of the bacteria spore fills the container (2) at a normal or low temperature, and the container (2) is sealed by a lid (3).

Accordingly, almost the virus except the bacteria spore in the container (2) is sterilized by the sterilizing agent (b), mold spore, i.e., a kind of ascomycete or like, which is hardly sterilized by the sterilizing agent (b), is sterilized by the synergetic effects of the sterilizing agent (b) and the heated water (c), and hence, only the bacteria spore held in bacteriostatic condition by the suppression of germination due to the acidity of the content (a) remains in the interior of the container (2), and accordingly, the content (a) can be reserved for a long term without becoming corrupted. In addition, since the bacteria spore remains alive, the sterilizing treatment can be simplified by, for example, reducing the using amount of the sterilizing agent, and manufacturing cost for the packaging container can be also correspondingly reduced. Furthermore, the interior of the container (2) can be cleaned simultaneously with the sterilization by the heated water (c), so that the sterilizing agent (b) can be prevented from remaining. Still furthermore, since the content (a) can fill the container (2) at a normal temperature, it is not necessary to locate any reinforcing rib, any pressure reducing panel and the like for the container (2), and accordingly, using amount of materials such as resin for making the container (2) can be extremely reduced. Still furthermore, it is not necessary to crystallize the mouth portion (2a) of the container (2), thus cheaply providing the packaging container (1, 28).

As recited in claim 2, in the packaging container (1, 28) recited in claim 1, the sterilizing agent is hydrogen peroxide, and the interior of the container is sterilized by blowing mist or gas (b) of the hydrogen peroxide into the container (2), as recited in claim 3, in the packaging container (1, 28) recited in claim 2, the mist (b) of the hydrogen peroxide to be fed into the container (2) is of an amount of 5 to 50 μL/container, or as recited in claim 4, in the packaging container (1, 28) recited in claim 2, the gas (b) of the hydrogen peroxide to be fed into the container (2) has a gas density of 1 to 5 mg/L to sterilize the interior of the container.

According to these examples, the inner surface of the container (2) can be evenly sterilized, and in addition, since it is not necessary to sterilize the bacteria spore, the using amount of the hydrogen peroxide can be reduced. A merit of using the 35% hydrogen peroxide in the state of mist or gas resides in that when the hydrogen peroxide evaporated at a high temperature contacts the container of a temperature lower than the dew point, it provides high density of about 70%, which then is condensed and adheres to the inner surface of the container (2). Further, this dew-condensing phenomenon is not affected by the shape of the container different from the case of spraying liquid.

As recited in claim 5, in the packaging container (1, 28) recited in claim 1, the heated water (c) has a temperature of 65 to 85° C. and is fed at a feeding rate of 5 to 15 L/min. Therefore, the packaging container (1, 28) in which other virus which is hardly sterilized by the sterilizing agent (b) such as ascomycete can be sterilized, and no hydrogen peroxide remains, is provided.

As recited in claim 6, in the packaging container (1, 28) recited in claim 1, the inner content (a) has an acidity of less than pH 4.6. According to this example, there is provided a packaging container (1, 28) capable of preventing the germination of the bacteria spore and preventing the corrosion of the content (a).

As recited in claim 7, in the packaging container (1, 28) recited in claim 1, the inner content (a) fills the container at a normal temperature of 3 to 40° C.

According to this example, it is not necessary to sterilize the bacteria spore, and there can be provided a packaging container (1, 28) which does not require for the content to fill the container in a state heated to a high temperature, to be reserved for a long term after the filling process and to be heated and sterilized from the external side of the container. Accordingly, the inner content (a) is hardly denatured, and in addition, the packaging container (1, 28), which does not require to form a reduced pressure absorption panel in consideration of deformation of the container (2) due to the heating or cooling of the same and not require to crystallize the mouth portion (2a) of the container (2).

As recited in claim 8, in the packaging container (1, 28) recited in claim 1, the inner content is a liquid beverage (a). Thus, the liquid drink (a) can be reserved for a long term at a normal temperature.

As recited in claim 9, in the packaging container (1, 28) recited in claim 1, the container (2) is made of PET (polyethylene telephthalate) material or polyethylene material. In this example, since the content can fill the container (2) at a normal temperature, it is not necessary to increase the heat-resisting performance of the container (2), and accordingly, the using amount of the resin material can be reduced, hence reducing the manufacturing cost of the container (2).

As recited in claim 10, in the packaging container (1, 28) recited in claim 1, the container is a bottle (2). In this example, the container can be easily treated.

The invention according to claim 11 is a method of manufacturing a packaging container, comprising the steps of: sterilizing an interior of a container (2) by a sterilizing agent (b) and a heated water (c) so as to allow bacterial spore to be alive but not to allow bacteria vegetative cell, mold and yeast to be alive; filling the container with an inner content (a), which has been subjected to a sterilization treatment, having an acidity suppressing germination of the bacteria spore at a normal or low temperature; and sealing the container (2) by a lid (3).

According to this method, it is not necessary to sterilize the bacteria spore, and the interior of the container (2) can be easily and speedily sterilized. In addition, almost the virus except the bacteria spore in the container (2) is sterilized by the sterilizing agent (b), other virus, which is hardly sterilized by the sterilizing agent (b), is sterilized by the heated water (c), and hence, only the bacteria spore held in bacteriostatic condition by the suppression of germination due to the acidity of the content (a) remains in the interior of the container (2), and accordingly, the content (a) can be reserved for a long term without becoming corrupted. Furthermore, the interior of the container (2) can be cleaned simultaneously with the sterilization by the heated water (c), so that the sterilizing agent (b) can be prevented from remaining. Still furthermore, since the content (a) can fill the container (2) at a normal temperature, it is not necessary to locate any reinforcing rib, any pressure reducing panel and the like for the container (2), and accordingly, the using amount of materials such as resin for making the container (2) can be extremely reduced. Still furthermore, it is not necessary to crystallize the mouth portion (2a) of the container (2), thus cheaply providing the packaging container (1, 28).

As recited in claim 12, in the manufacturing method of the packaging container (1, 28) recited in claim 11, the interior of the container (2) is sterilized by blowing mist or gas (b) of a hydrogen peroxide as sterilizing agent into the container (2), as recited in claim 13, in the manufacturing method of the packaging container (1, 28) recited in claim 11, the mist (b) of the hydrogen peroxide to be fed into the container (2) is of an amount of 5 to 50 μL/container, or as recited in claim 14, in the manufacturing method of the packaging container (1, 28) recited in claim 11, the gas (b) of the hydrogen peroxide to be fed into the container (2) has a gas density of 1 to 5 mg/L. According to these examples, the inner surface of the container (2) can be evenly sterilized, and furthermore, since the bacteria spore is allowed to remain alive, the using amount of the hydrogen peroxide can be reduced.

As recited in claim 15, in the manufacturing method of the packaging container (1, 28) recited in claim 11, the heated water (c) has a temperature of 65 to 85° C. and is fed at a feeding rate of 5 to 15 L/min. According to this example, other virus which is hardly sterilized by the sterilizing agent (b) for the ascomycete or like bacteria can be sterilized, and in addition, the hydrogen peroxide can be prevented from remaining in the container.

As recited in claim 16, in the manufacturing method of the packaging container (1, 28) recited in claim 11, the inner content (a) has an acidity of less than pH 4.6. In this example, the germination of the bacteria spore during the reservation of the content (a) can be blocked, thus preventing the content (a) from becoming corrupted.

As recited in claim 17, in the manufacturing method of the packaging container (1, 28) recited in claim 11, the inner content (a) fills the container at a normal temperature of 3 to 40° C.

According to this example, it is not necessary to sterilize the bacteria spore, and accordingly, it does not require for the content to fill the container in a state heated to a high temperature, to be reserved for a long term after the filling process and to be heated and sterilized from the external side of the container. Accordingly, the inner content (a) is hard to be denatured, and in addition, it does not require to form a reduced pressure absorption panel in consideration of deformation of the container (2) due to the heating or cooling of the same and not require to crystallize the mouth portion (2a) of the container (2).

As recited in claim 18, in the manufacturing method of the packaging container (1, 28) recited in claim 11, the inner content (a) is a liquid beverage. Thus, the liquid container (a) can be reserved for a long term.

As recited in claim 19, in the manufacturing method of the packaging container (1, 28) recited in claim 11, the container (2) is made of PET (polyethylene telephthalate) material or polyethylene material. According to this example, the using amount of the PET or polyethylene material can be reduced, thus reducing the manufacturing cost of the container (2).

As recited in claim 20, in the manufacturing method of the packaging container (1, 28) recited in claim 11, the container is a bottle (2). Thus, the container (2) can be easily handled.

As recited in claim 21, in the manufacturing method of the packaging container (1, 28) recited in claim 11, the bottle (2) is subjected to a blow-molding process from a preform (10) just before the sterilization treatment of the interior of the bottle (2). According to this example, it is possible to convey the container (2) in form of a preform (10) having a small volume in comparison with a bottle having a large volume to a portion at which the packaging container (1, 28) is manufactured, whereby the conveying cost can be reduced, resulting in the reduction of the manufacturing cost of the packaging container (1, 28).

As recited in claim 22, in the manufacturing method of the packaging container (1, 28) recited in claim 11, an exterior of the container (2) is sterilized by the sterilizing agent (b) and the interior of the container (2) is sterilized by the heated water (c) in a state in which the sterilizing agent (b) adheres to an outer surface of the container (2). According to this example, the container (2) is introduced into the aseptic filling machine after the outer surface of the container (2) has been sterilized, and therefore, the contamination by bacteria or like in the interior of the aseptic filling machine at the time of manufacturing the packaging container can be prevented. In addition, since the heated water (c) is fed into the container (2) with the sterilizing agent (b) adhering to the outer surface of the container (2), the sterilizing performance outside the container (2) can be improved in accordance with the increasing of the temperature of the outside of the container (2).

The invention according to claim 23 is an apparatus for manufacturing a packaging container (1, 28) comprising: a conveying unit that conveys a container (2) along a predetermined conveying root; a first sterilizing unit (5) that is disposed along the conveying root for performing the sterilization of an interior of the container (2) by a sterilizing agent (b) so as to allow bacterial spore to be alive but not to allow bacteria vegetative cell, mold and yeast to be alive; a second sterilizing unit (6) that performs the sterilization of an interior of the container (2) by a heated water (c) so as to allow bacterial spore to be alive but not to allow bacteria vegetative cell, mold and yeast to be alive; a content filling unit (7) that fills the container (2) with an inner content (a), which has been subjected to a sterilization treatment, having an acidity suppressing germination of the bacteria spore at a normal or low temperature; and a sealing unit (8) that seals the container (2) by a lid (3), wherein the respective units are arranged in the described order, and a portion between the first sterilizing unit (5) to the sealing unit (8) is covered by aseptic chambers (23, 24, 26, 27).

According to this aspect, it is not necessary to sterilize the bacteria spore, and in addition, the packaging container (1, 28), the interiors of the aseptic chambers (23, 24, 26, 27) surrounding the packaging container and the interior of the container (2) can be easily and speedily sterilized. Therefore, the packaging container manufacturing apparatus can be made compact and simplified.

Furthermore, almost the virus except the bacteria spore in the container (2) is sterilized by the sterilizing agent (b) in the first sterilizing unit (5), other virus, which is hardly sterilized by the sterilizing agent (b), is sterilized by the synergistic effects of the first and second sterilizing units (5) and (6), and hence, only the bacteria spore held in bacteriostatic condition by the suppression of germination due to the acidity of the content (a) remains in the interior of the container (2), and accordingly, the content (a) can be reserved for a long term without becoming corrupted.

Still furthermore, the interior of the container (2) can be cleaned simultaneously with the sterilization by the heated water (c), so that the sterilizing agent (b) can be prevented from remaining.

Still furthermore, since the content (a) can fill the container (2) at a normal temperature, it is not necessary to locate any reinforcing rib, pressure reducing panel and the like for the container (2), and accordingly, using amount of materials such as resin for making the container (2) can be extremely reduced.

Still furthermore, it is not necessary to crystallize the mouth portion (2a) of the container (2), thus cheaply providing the packaging container (1, 28).

Still furthermore, since the first sterilizing unit (5) is covered by the aseptic chamber (23), the mist or gas of the sterilizing agent fills in a supersaturated state in the aseptic chamber (23), and accordingly, the virus entering the aseptic chamber (23) in a state of adhering to the container (2) or together with air-flow caused by the travelling container (2) can be speedily and securely sterilized by the condensation of the mist or gas of the sterilizing agent into hydrogen peroxide water of high density. Accordingly, the packaging container in which the aseptic condition inside the aseptic chambers (23, 24, 26, 27) can be highly maintained and which is superior in the aseptic performance can be manufactured.

As recited in claim 24, in the apparatus for manufacturing a packaging container (1, 28) recited in claim 23, the sterilizing unit is composed of a nozzle (5) for blowing mist or gas (b) of hydrogen peroxide as the sterilizing agent into the container (2), and the nozzle has a front end facing a mouth portion (2a) of the container (2). According to this example, the mist or gas (b) of the hydrogen peroxide can be effectively supplied into the container now in travelling.

As recited in claim 25, in the apparatus for manufacturing a packaging container (1, 28) recited in claim 23, the container is a bottle (2) and a blow-molding unit (9) that molds the bottle from a preform (10) is disposed just before the sterilizing unit. According to this example, since the blow-molding unit (9) is disposed on the upstream side of the packaging container manufacturing apparatus, it is possible to convey the preform (10) having a small volume in comparison with a bottle (2) to the manufacturing apparatus for the packaging container (1, 28), whereby the conveying cost can be reduced, resulting in the reduction of the manufacturing cost of the packaging container (1, 28).

As recited in claim 26, in the method of manufacturing a packaging container (1, 28) recited in claim 11, with respect to a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive, the interior of the product line is positively pressurized, thereafter, the interior of the product line is cooled to a normal temperature by passing an aseptic water or the content, and the content is fed to the container through the thus cooled product line.

According to this example, the product line, in which the acidic beverage having relatively high acidity and allowing the spore to remain but not suitable for a hot-pack method fills the packaging material such as container, can be easily sterilized for a short time. Therefore, the packaging line, in which the content fills the container, can be speedily operated, hence increasing the manufacturing efficiency.

As recited in claim 27, in the method of manufacturing a packaging container (1, 28) recited in claim 26, the heated water has a temperature of 80 to 140° C. and passes through the product line for 1 to 30 minutes. In this example, the product line can be sterilized with a proper thermal efficiency.

As recited in claim 28, in the apparatus for manufacturing a packaging container (1, 28) recited in claim 23, the apparatus further comprising: a heating and sterilizing unit that sterilizes and positively pressurizes an interior of a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive; and a cooling unit that cools the interior of the product line to a normal temperature by passing an aseptic water or content through the product line after the sterilization by the heating and sterilizing unit.

According to this example, the product line, in which the acidic beverage having relatively high acidity and allowing the spore to remain fills the packaging material such as container, can be easily sterilized for a short time. Therefore, the packaging line, in which the content fills the container, can be speedily operated, hence increasing the manufacturing efficiency.

As recited in claim 29, in the apparatus for manufacturing a packaging container (1, 28) recited in claim 28, the heated water has a temperature of 80 to 140° C. and passes through the product line for 1 to 30 minutes. In this example, the product line can be sterilized with a proper thermal efficiency.

As recited in claim 30, in the apparatus for manufacturing a packaging container (1, 28) recited in claim 28, the inner content has an acidity of less than pH 4.6. According to this example, it is only sufficient to suppress the germination of the spore, the temperature, the flow rate, the supplying time and the like of the heated water to be fed to the product line can be suitably shortened, and accordingly, the cooling to the normal temperature thereafter can be smoothly performed to thereby smoothly start the filling working.

As recited in claim 31, in the method of manufacturing a packaging container (1, 28) recited in claim 11, steps of sterilizing the interior of the container (2), filling the container with the content (a), and sealing the container with the lid (3) are performed inside the aseptic chambers (23, 24, 26, 27), the interiors of the aseptic chambers being preliminarily sterilized by the sterilizing agent and the heated water so as to allow bacterial spore to be alive but not to allow bacteria vegetative cell, mold and yeast to be alive, or as recited in claim 32, in the apparatus for manufacturing a packaging container (1, 28) recited in claim 23, the first sterilizing unit (5), the second sterilizing unit (6), the content filling unit (7) and the sealing unit (8) are covered by the aseptic chambers (23, 24, 26, 27), in which nozzles (78, 29) are disposed so as to inject the sterilizing agent and the heated water successively, and by successively injecting the sterilizing agent and the heated water through the nozzles, the interior of the aseptic chambers (23, 24, 26, 27) are preliminarily sterilized so as to allow bacterial spore to be alive but not to allow bacteria vegetative cell, mold and yeast to be alive, the using amounts of the utilities such as the sterilizing agent, the heated water, the hot-air and the like can be reduced. Therefore, the manufacturing cost of the packaging container can be also reduced, and the manufacturing of the packaging container can speedily start.

### BRIEF DESCRIPTION OF THE DRAWINGS

FIG. 1 is a front view representing one embodiment of a packaging container according to the present invention.

FIG. 2 is a flowchart explaining one embodiment of a packaging container manufacturing method according to the present invention.

FIG. 3 is a view explaining processes in respective steps shown in the flowchart of FIG. 2.

FIG. 4 is a schematic plan view representing one embodiment of a packaging container manufacturing apparatus according to the present invention.

FIG. 5 is a diagrammatic elevational view, partially cut away, of one example of a hydrogen peroxide gas generator.

FIG. 6 is a schematic plan view representing another embodiment of a packaging container manufacturing apparatus according to the present invention.

FIG. 7 is a diagrammatic elevational view, partially cut away, of another example of a hydrogen peroxide gas generator.

FIG. 8 is a front view representing another embodiment of a packaging container according to the present invention.

FIG. 9 is a block diagram showing one example of a product line in a packaging container manufacturing apparatus of the present invention.

FIG. 10 is a block diagram showing one example of a packaging line connected to the product line shown in FIG. 9.

FIG. 11 is an illustrated plan view showing a sterilizing apparatus in the aseptic chamber in the packaging container manufacturing apparatus according to the present invention.

FIG. 12 is a sectional view taken along the line XII-XII in FIG. 11.

### EXPLANATION OF REFERENCE NUMERALS

1, 28—packaging container, 2—bottle, 2a—bottle mouth portion, 3—cap, 5, 6, 7—nozzle, 8—capper, 10—preform, 23, 24, 26, 27—aseptic chamber, a—beverage (drink), b—hydrogen peroxide mist or gas, c—heated water, 76—conduit (duct), 77—returning conduit, 78—spray nozzle of sterilizing agent, 79—spray nozzle for heated water.

### BEST MODE FOR EMBODYING THE INVENTION

Hereunder, the best mode of the present invention will be described with reference to the accompanying drawings.

### Embodiment 1

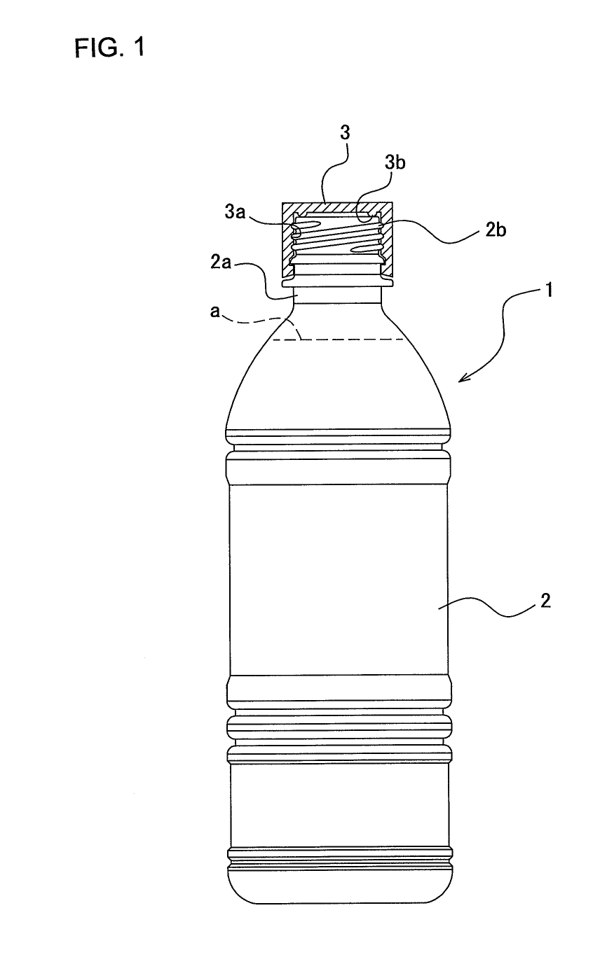

As shown in FIG. 1, the packaging container or body 1 includes a bottle 2 as a container and a cap 3 as a lid. A male thread 2b is formed to a mouth portion 2a of the bottle, and a female thread 3a is also formed to the cap 3, and the mouth portion 2a of the bottle 2 is sealed through engagement of the male and female threads 3a and 2b.

The bottle 2 is formed through a blow-formation or molding of a preform made of PET, not shown, having approximately test tube shape. The bottle 2 may be formed of another resin material such as polypropylene, polyethylene or other without being limited to PET resin. The preform is molded through an injection molding process or like and is provided with an approximately test tube-shaped body and a mouth portion 2a of the bottle 2. The mouth portion 2a is formed with a male thread 2b at the same time as the preform injection molding time. The cap 3 is formed of a resin material such as polypropylene or like through an injection molding process, and a female thread 3a is also formed at the same time as the cap molding time.

The interior of the bottle 2 is sterilized by using sterilizing agent and heated water, before the filling of liquid beverage (drink) as inner content, so as to allow the spore of bacteria to remain alive, but not to allow bacteria vegetative cells, molds and yeasts to remain alive.

As the sterilizing agent, for example, hydrogen peroxide will be utilized. In the use thereof, mist or gas of the hydrogen peroxide is generated and then introduced into the bottle 2 through the mouth portion 2a. As mentioned, the interior of the bottle 2 is sterilized by the hydrogen peroxide mist or gas, the inside surface of the bottle 2 can be evenly sterilized with small amount of hydrogen peroxide to be used.

Since it is not necessary to sterilize the spore of the bacteria, the use amount of the hydrogen peroxide will be reduced. For example, 5 to 50 μL (micro-litter)/bottle of the hydrogen peroxide will be used. In a case of using the hydrogen peroxide gas, the gas density is 1 to 5 mg/L.

Furthermore, the heated water is fed to the bottle 2 at a temperature of 65 to 75° C. at flow rate of 5 to 10 L/min. By the feeding of the heated water into the bottle 2, the mold spore of ascomycete or like which is hardly sterilized by the hydrogen peroxide but relatively weak to heat. In addition, when the heated water cleans the interior of the bottle 2, the hydrogen peroxide can be prevented from remaining inside.

Although the spore of the bacteria remains in the bottle 2 in the living state (alive), the sterilized beverage “a” having acidity capable of preventing growing of spore of bacteria fills the bottle 2, thus the beverage can be prevented from being denatured and becoming corrupted. It is desirable for the beverage to have acidity of less than pH 4.6, and more preferably, less than pH 4. The beverage having pH 4.6 to pH 4 includes tomato juice, vegetable juice, or like, and the beverage having pH less than 4.6 includes lemon tea, orange juice, lactic carbonate drink, functional drink, lemon juice with carbonate, grape juice, fruit juice or like.

Further, this beverage “a” fills the bottle 2 at normal temperature. The beverage “a” is preliminarily sterilized by being heated or like and then cooled at the normal temperature of 30 to 40° C., which then fills the bottle 2.

As mentioned above, since the spore of the bacteria is allowed to remain alive inside the bottle 2, it is not necessary for the beverage “a”, as has been required in the prior art to fill the bottle 2 in the heated state to high temperature, to remain for a long time after the filling in the bottle 2 or to heat the packaging container 1 filled up with the beverage and closed with the cap 3 from the outside thereof so as to sterilize the beverage. Accordingly, the beverage as inner content is hardly denatured, and it is not necessary to form a pressure reduction absorption panel in consideration of the deformation of the bottle 2 due to heating or cooling of the beverage “a” or to crystallize the mouth portion 2a of the bottle 2.

The mouth portion 2a of the bottle 2 is closed with the cap 3 and then sealed so as to prevent external air or virus from invading into the bottle 2. As mentioned above, since the beverage “a” fills the bottle 2 at the normal temperature, the mouth portion 2a of the bottle 2 is not deformed by the heat. Accordingly, ribs 3b of the cap 3 is properly tightly contacted to the mouth portion 2a of the bottle 2 to thereby seal the bottle 2 for a long time.

As mentioned above, only the spore of the bacteria remains inside the bottle 2, and the spore of the bacteria is restricted from growing by the acidity of the beverage “a” and is maintained in bacteriostatic condition. Thus, the beverage can be prevented from being corrupted for a long time and can be preserved under the normal temperature in a suitable condition, thereby producing the packaging container 1 as commercially aseptic product.

A manufacturing method of the packaging container of the structure mentioned above will be described hereunder.

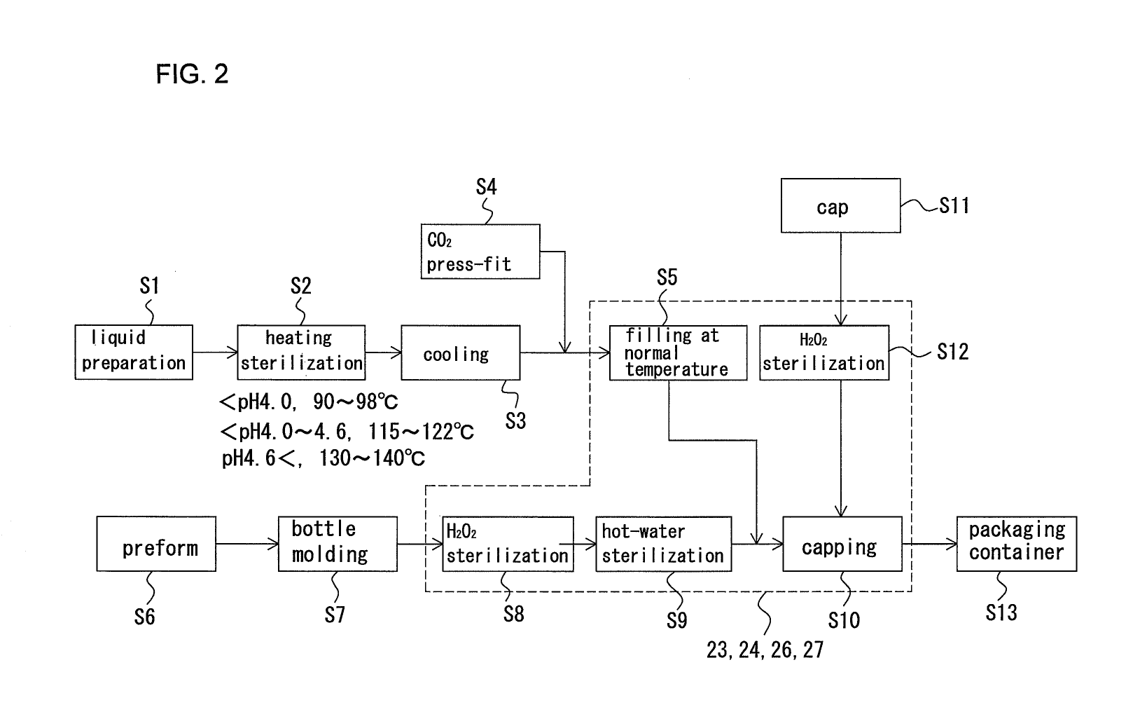

As shown in FIG. 2, the beverage “a” as inner content is made up or prepared (step S1), and a heating sterilization process is performed (step S2). In this heating process, the heating temperature is set to be about 90 to 98° C. at pH 4.0 of the acidity of the beverage “a”, and about 115 to 122° C. at pH 4.0 to pH 4.6. According to this process, virus which may be grown in the packaging container in the beverage “a” before the filling thereof can be completely sterilized.

The beverage “a” after the heating sterilization process is then cooled to the normal temperature of 30 to 40° C. (step S3). This cooling may be performed through heat exchanging process between the heated beverage “a” and a beverage before the heating.

On the other hand, a preform is prepared (step S6), and the bottle 2 is formed from the preform by the blow-molding machine (blow-molder) (step S7). The bottle 2 may be formed from polypropylene, polyethylene or other resin material as well as PET.

The sterilization process is performed by the hydrogen peroxide and heated water to the inside surface of the bottle 2, and the sterilizing process by the hydrogen peroxide is also performed to the outside surface thereof (steps S8 and S9).

Further, the steps from the preform supply (step S6) to the sterilization processing (step S8) through the bottle formation (step S7) may be performed independently at different portions, but it is desirable that these steps are continuously performed. By continuously performing these steps, the packaging container can be conveyed to the packaging container manufacturing site in form of the preform having relatively small volume without taking in form of the bottle 2 having relatively large volume, which reduces conveying cost, resulting in the reduction of the manufacturing cost of the packaging container 1.

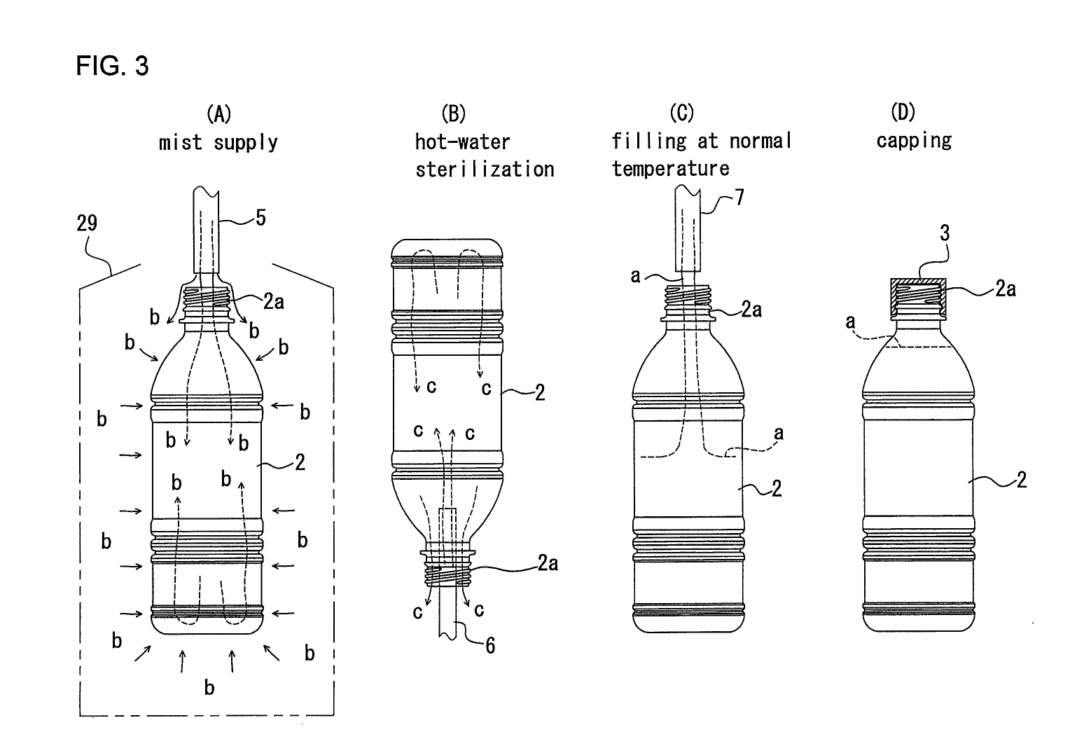

The hydrogen peroxide is deformed into mist by a hydrogen peroxide gas generator 4, and as shown in FIG. 3(A), this mist is jetted toward the bottle 2 from the nozzle 5. The opening of the nozzle 5 faces the opening of the mouth portion 2a of the bottle 2 with a distance, and the mist “b” jetted from the nozzle 5 flows into the bottle 2. The mist “b” adheres to the entire inner surface of the bottle 2 to thereby sterilize the bacteria vegetative cells, molds and yeasts within the bottle 2. The amount of the mist “b” of the hydrogen peroxide to be supplied into the bottle 2 is 5 to 50 μL/bottle, and is an amount capable of sterilizing the bacteria vegetative cells, molds and yeasts, but not sterilizing the spores of bacteria, whereby the amount of the hydrogen peroxide to be used may be reduced.

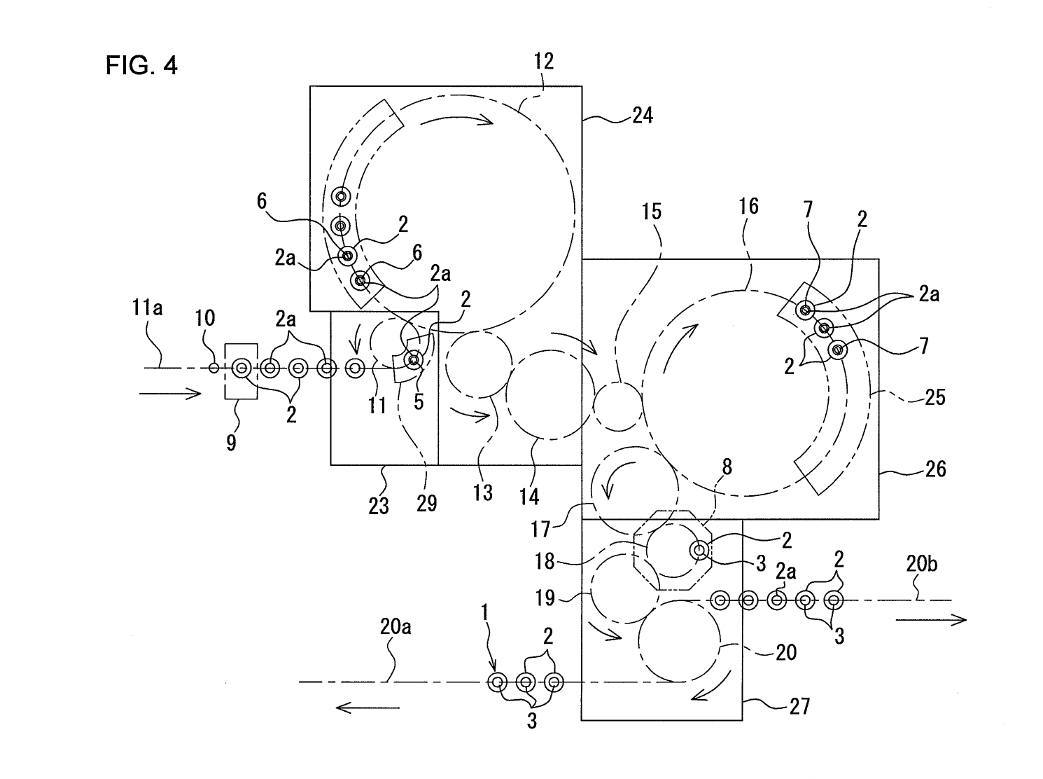

Furthermore, as shown in FIG. 3(A) and FIG. 4, a tunnel 29 is arranged in the vicinity of the nozzle 5 so as to surround the bottle 2 positioned below the nozzle 5. The hydrogen peroxide mist or gas of high density stays inside the tunnel 29. Because of this reason, the hydrogen peroxide mist or gas “b” can adhere to the entire outer surface of the bottle 2 to thereby sterilize the vegetative cells, molds and yeasts adhering to the outer surface of the bottle 2. As mentioned, since the outer surface of the bottle 2, the invasion of the vegetative cells, molds and yeasts adhering to the outer surface of the bottle 2 into the bottle 2 and the filling machine in the aseptic condition can be prevented, and hence, the beverage “a” filling the bottle 2 can be also prevented from being contaminated.

The bottle 2 of which the inner and outer surfaces are sterilized by the hydrogen peroxide as the sterilizing agent is thereafter subjected to the sterilization of the heated water (step S9). More specifically, as shown in FIG. 3(B), the heated water of the temperature of 65 to 75° C. is supplied into the bottle 2 through the nozzle at a flow rate of 5 to 10 L/min. At this time, it is desirable that the bottle 2 takes an inverted attitude, and the nozzle 6 is inserted into the bottle 2 through the downwardly opened mouth portion 2a toward a shoulder portion thereof. The heated water “c” flowing into the bottle 2 circulates in the bottle 2 and then flows out of the bottle 2 through the mouth portion 2a thereof. This heated water “c” sterilizes the mold as a part of ascomycete damaged by the hydrogen peroxide. In addition, the excessive hydrogen peroxide remaining in the bottle 2 is washed out by the heated water “c” outside the bottle 2.

Here, at the time when the inner surface of the bottle 2 is sterilized by the heated water “c”, although the hydrogen peroxide mist “b” may adhere to the outer surface of the bottle 2, the sterilizing performance to the outer surface of the bottle 2 can be enhanced by the heat transfer of the heated water “c” to the outside of the wall surface thereof.

The beverage “a” sterilized by the heated water and cooled to the normal temperature fills the bottle 2 which has been sterilized by the heated water “c” at the normal temperature (step S5). The beverage “a” has a temperature of about 30 to 40° C. at the filling time. As mentioned above, according to the manufacturing method of this packaging container 1, since it is not necessary to sterilize the spore of the bacteria, it is also not necessary to fill the bottle 2 with the beverage “a” in a highly heated state, to reserve it for a long time after the filling and to heat and sterilize the packaging container 1 from the outside thereof. Therefore, the beverage “a” is hardly deformed and it is also not necessary to provide a pressure reduction absorption panel in consideration of the possibility of deformation of the bottle 2 due to cooling and to crystallize the mouth portion 2a of the bottle 2.

The filling of the beverage “a” is performed, specifically as shown in FIG. 3(C), by placing the nozzle 7 to face the mouth portion 2a of the bottle 2 and draining the beverage “a” from the nozzle 7. As mentioned above, the acidity of this beverage “a” is desirably to be less then pH 4.6, and more preferably, less than pH 4.0, and it is possible to fill the bottle 2 with tomato juice, vegetable juice, lemon tea, orange juice, lactic carbonate drink, functional drink, carbonate lemon juice, grape juice, fruit juice and the like. That is, according to this manufacturing method, the packaging containers for almost all the beverages or drinks except for barley tea or milked drink can be manufactured. Of course, not to say, it is also possible to manufacture the packaging containers for carbonate drinks such as Coca-Cola, cider or like including no animal or vegetable components and having carbon gas pressure of more than 1.01 g/cm² (20° C.).

At the time when the beverage fills the bottle, since the outer surface of the bottle 2 is preliminarily sterilized, any virus is not drawn into the bottle 2 together with the beverage “a”, and the contamination by the molds of the beverage “a” can be preferably prevented.

The bottle 2 filled with the predetermined amount of the beverage “a” is then sealed by the cap 3 as shown in FIG. 3(D) (step S10). A number of caps 3 are preliminarily collected (step S11) and arranged and conveyed in a row so as to face the bottles 2 which is to be filled with the beverage “a”, and the hydrogen peroxide mist “b” is sprayed onto the inner and outer surfaces of the bottles 2 during the conveyance, thus performing the sterilization process thereto (step S12). Thereafter, each of the caps 3 are applied to the mouth portion 2a of each bottle 2 and then fastened through screw engagement, for example.

As the sterilizing method for the cap 3, the method disclosed in the Japanese Patent Publication No. 3778952 may be adopted, for example.

Further, the process from at least the beverage filling step at the normal temperature (step S5) to the capping step (step S10) is performed under the aseptic environment (atmosphere) surrounded by the aseptic chambers. The interiors of these aseptic chambers are sterilized by preliminarily spraying the hydrogen peroxide or applying the heated water so as to allow the spore of the bacteria to remain alive but not to allow the bacteria vegetative cells, molds and yeasts to remain alive in the aseptic chambers. After the aseptic sterilization treatment, the aseptic air of positive pressure is supplied so that the aseptic air is always blown toward the exterior of the aseptic chamber.

The capped bottles 2 are discharged from the manufacturing process as products of packaging containers 1 (step S13).

Next, one example of the manufacturing apparatus for carrying out the manufacturing method for the packaging container 1 of the characters mentioned above will be described hereunder.

As shown in FIG. 4, the manufacturing apparatus includes means for conveying the PET bottles 2 mentioned above along a predetermined conveying path or line.

The conveying means is constructed such that a plurality of wheels 11, 12, 13, 14, 15, 16, 17, 18, 19, 20 are disposed horizontally in an adjacent arrangement and a plurality of grippers, not shown, are also arranged at predetermined pitch around these wheels 11, 12, 13, 14, 15, 16, 17, 18, 19, 20. These wheels 11, 12, 13, 14, 15, 16, 17, 18, 19, 20 are of course added or reduced in numbers as occasion demands. The adjacent wheels are rotated at the same peripheral speed in directions reverse to each other, and the grippers are turned at the same peripheral speed around the respective wheels 11, 12, 13, 14, 15, 16, 17, 18, 19, 20. The conveying path of the conveying means extends in a continuous circular-arc shape by connecting the respective wheels 11, 12, 13, 14, 15, 16, 17, 18, 19, 20, and a number of bottles 2 travel at the predetermined interval on the continuous circular-arc line. That is, the bottle 2 is turned in a state held by the gripper of the upstream-side wheel together with the wheel, and when the bottle 2 reaches the downstream-side wheel, the bottle 2 is transferred to the gripper of the downstream-side wheel. This transferring process is subsequently performed toward the downstream side at the constant speed.

Further, since a known gripper and its opening/closing mechanism may be used, details thereof are omitted herein.

As shown in FIG. 4, the nozzle 5 (FIG. 3(A)) as the first sterilizing means for sterilizing the interior of the bottle 2 by the hydrogen peroxide as sterilizing agent, the nozzle 6 (FIG. 3(B)) as the second sterilizing means for sterilizing the interior of the bottle 2 by injecting the heated water “c” therein, the nozzle 7 (FIG. 3(C)) as the filling means for filling the sterilized bottle 2 at the normal temperature with the beverage “a” as inner content, the capper 8 (FIG. 3(D)) as the sealing means for sealing the bottle 2 with the cap 3 as a lid, are sequentially arranged in the described order along the conveying path.

Furthermore, although the bottle outer surface sterilizing means for sterilizing the outer surface of the bottle 2 by the hydrogen peroxide mist “b” is also arranged along the above-mentioned conveying path, in the embodiment 1, the nozzle 5 as the first sterilizing means is operated also as this bottle outer surface sterilizing means.

An introduction conveyer 11a is connected on the upstream-side of the first wheel 11 to which the nozzle 5 as the first sterilizing means, and the blow-forming (-molding) machine 9 is installed on the introduction conveyer 11a. The perform 10 is supplied to the blow-forming machine 9, and the bottle 2 molded from the preform 10 by the blow-forming machine 9 is conveyed to the first wheel 11 at the constant pitch.

One or a plurality of (at least one) nozzles 5 of the first sterilizing means may be arranged. The nozzle 5 has an end opening directly facing the opening of the mouth portion 2a of the bottle 2 with a predetermined distance. The hydrogen peroxide mist “b” discharged from the opening of the nozzle 5 flows into the bottle 2 through the mouth portion 2a of the bottle 2 as shown in FIG. 3(A).

Furthermore, in the first wheel 11, a tunnel 29 is provided at a portion, below the nozzle 5, through which the bottle 2 passes. A part of the hydrogen peroxide mist “b” discharged from the nozzle opening fills the interior of the tunnel 29 and adheres to the outer surface of the bottle 2, thus effectively sterilizing the outer surface of the bottle 2.

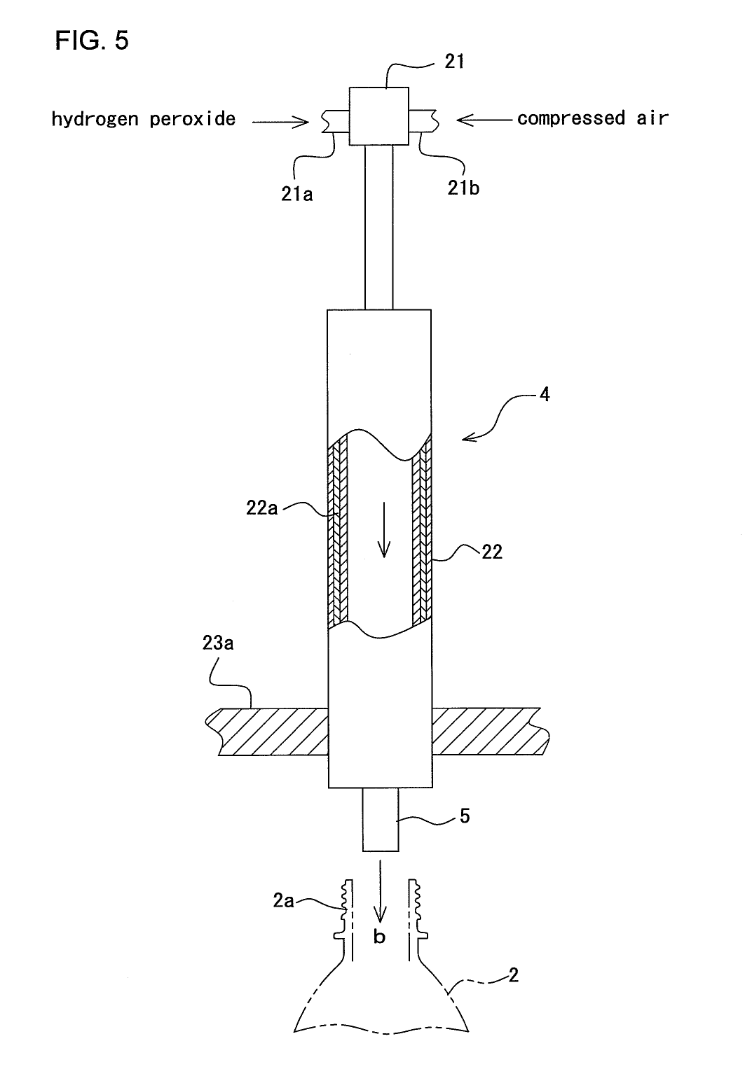

The hydrogen peroxide mist “b” is generated by a hydrogen peroxide gas generating device 4, for example, shown in FIG. 5. This generating device 4 is provided with a hydrogen peroxide supply section 21 as a two-fluid spray for supplying the solution of the hydrogen peroxide as sterilizing agent in form of drops and an evaporating (vaporizing) section 22 for heating the mist of the hydrogen peroxide supplied from the hydrogen peroxide supply section 21 to a temperature less than non-decomposition temperature more than boiling temperature of the hydrogen peroxide. The hydrogen peroxide supply section 21 takes solution and compressed air of the hydrogen peroxide from a hydrogen peroxide supply path 21a and a compressed gas supply path 21b, and then, the hydrogen peroxide solution is atomized into the evaporating section 22. The evaporating section 22 is composed of a pipe in which a heater 22a is sandwiched between inner and outer walls thereof, and the hydrogen peroxide mist blown into the pipe is heated and evaporated. The thus evaporated hydrogen peroxide gas is jetted out toward the mouth portion 2a of the bottle 2 from the nozzle 5. The temperature of the evaporated hydrogen peroxide is lowered to a temperature below its boiling point till the time of reaching in a vicinity of the bottle from the nozzle 5, and a part of the hydrogen peroxide is condensed and liquefied. Accordingly, fine mist as the gas-liquid mixture of the hydrogen peroxide is generated, Such fine hydrogen peroxide mist is blown into the interior of the bottle 2 from the nozzle 5, thus adhering to the entire inner surface of the bottle 2. The mist adhering to the entire inner surface of the bottle 2 is condensed into the hydrogen peroxide of high density, which effectively sterilizes the inner surface of the bottle 2.

The supply amount of this mist “b” is smaller in amount than that in a conventional aseptic method. By this mist “b”, although the bacteria vegetative cells, molds, yeasts and the like in the bottle 2 are sterilized, the spores of the bacteria still remain alive.

The first aseptic chamber 23 surrounds the first wheel 11 so as to enclose the nozzle 5 of the first sterilizing processing device mentioned above. The mist “b” discharged from the nozzle 5 fills inside the first chamber 23, and this mist is blown out of the entrance/exit port of the bottle 2 of the first aseptic chamber 23 together with the aseptic air also supplied into the first aseptic chamber 23, thus preventing the external atmosphere including virus from entering the chamber 23. The nozzle 5 is coupled with the first aseptic chamber 23 by means of a coupling member 23a, thereby being secured to a predetermined position in the first aseptic chamber 23.

In the first aseptic chamber 23, as shown in FIG. 3(A), the hydrogen peroxide of high density stays in the tunnel 29, and accordingly, even if the mist “b” discharged from the nozzle 5 flows externally of the bottle 2, the mist can sterilize the bacteria vegetative cells, molds, yeasts and the like existing and floating inside the bottle or those adhering to the inner surface of the bottle 2.

The gripper disposed around the outer periphery of the second wheel 12 is supported by the second wheel 12 side through a horizontal pivot, not shown, and contacts the cam curved in a circular shape with the swiveling shaft of the second wheel being the center of this swiveling motion. According to this motion, the bottle 2 is received from the contact point to the first wheel 11 and advances, and is then inverted in a vertical attitude. Thus, as shown in FIG. 3(B), the bottle 2 is also inverted vertically with its mouth portion 2a being directed downward.

As to the nozzle 6 of the second sterilizing processing device, one or more nozzles 6 are arranged so as to direct upward for supplying heated water “c” into the bottle 2, which is now directed downward as shown in FIG. 3(B). The nozzle 6 is disposed directly below each gripper so as to be swivel or turn together with the gripper. Though not shown, the respective nozzles 6 are moved vertically by the cam mechanism at the positions just below the corresponding grippers to be capable of freely entering the bottles 2. Further, the second sterilizing processing device supplies the aseptic heated (hot) water to the nozzles 6 via a manifold, hollow tubes and the like. As shown in FIG. 3(B), the heated water “c” blowing out from the nozzle 6 circulates in the bottle 2 and, thereafter, flows out of the bottle 2 through the mouth portion 2a thereof. The sterilization of the respective bottles 2 by the heated water is performed within the area indicated with tow-dot-chain line around the wheel 12 shown in FIG. 4.

This heated water “c” is excessively heated under the sterilizing condition in which the mold and yeast can be sterilized but the spore is not sterilized, thereafter, cooled to a temperature of 65 to 75° C., and then supplied through each nozzle 6 at flow rate of 5-10 L/min to each bottle 2. Further, the heated water may be produced by filtrating and sterilizing hot water by means of the filter and then increasing heat by means of heat exchanger. By this heated water, “c”, the mold, such as one kind of ascomycete, damaged by the hydrogen peroxide is sterilized. In addition, by this heated water “c”, the excessive hydrogen peroxide remaining in the bottle 2 is washed and discharged out of the bottle 2. Furthermore, the sterilizing effect to the outer surface of the bottle 2 can be enhanced by the hydrogen peroxide adhering to the outer surface of the bottle 2 due to the heat of the heated water “c”.

As shown in FIG. 4, the second, third and fourth wheels 12, 13 and 14 are surrounded by the second aseptic chamber 24 so as to include the nozzles 6 of the second sterilizing processing means. The aseptic air under positive pressure is also supplied in this second aseptic chamber 24.

The bottle 2 subjected to the sterilization treatment by the heated water “c” is transferred to the sixth wheel 16 from the second wheel 12 via third, fourth and fifth wheels 13, 14 and 15. The filler (filling machine) 25 is disposed to a predetermined position of this sixth wheel 16. The bottle 2 is conveyed, while being held by the gripper of the sixth wheel 16, and is filled up with the beverage (drink) “a” as inner content by the filler 25. The filler 25 is provided with a nozzle 7, as shown in FIG. 3(C), from which the predetermined amount of the beverage “a” is supplied so as to fill the bottle 2. One or plural nozzles 7 may be provided for the filler 25.

The beverage “a” is maintained at a normal (room) temperature of 30-40° C. at the filling time. Further, it is desirable for the beverage “a” to have acidity of less than pH 4.6, more desirably, less than pH 4. As such drink “a”, tomato juice, vegetable juice, lemon tea, orange juice, lactic carbonated drink, functional drink, carbonated lemon juice, grape juice, fruit juice or like may be provided.

A portion or area ranging from the fifth wheel 15 to the seventh wheel 17 is surrounded by the third aseptic chamber 26 so as to enclose the nozzle 7 of the filler 25. Aseptic air under positive pressure is also supplied into this third aseptic chamber 26.

As shown in FIG. 4, the capper 8 as the sealing means is disposed at the predetermined position of the eighth wheel 18. When the bottle 2 filled with the beverage “a” reaches the capper 8, the cap 3 is screwed and fastened to the mouth portion 2a of the bottle 2 as shown in FIG. 3D.

The peripheral portions of the eighth, ninth and tenth wheels 18, 19 and 20 are covered by the fourth aseptic chamber 27 so as to include the capper 8 therein. Positive aseptic air is also fed into this aseptic chamber 27.

The bottle 2 of which the mouth portion 2a is closed by the cap 3 is carried out externally of the fourth aseptic chamber 27 from the carry-out conveyer 20a through the tenth wheel 20 for rejecting the bottle 2, and then, the bottle 2 is shipped as a product of packaging container 1. On the other hand, the bottle 2 is judged as defective one for filling, capping or like process is discharged out externally of the fourth aseptic chamber 27 through a root other than that mentioned above from the conveyer 20b for rejection and then recovered.

The mist “b” discharged from the nozzle 5 fills the interior of the first aseptic chamber 23. As a matter of fear, in the interior of this first aseptic chamber 27, the virus may adhere to the bottle 2 or enter the aseptic chambers 23, 24, 26 and 27 with air-flow caused by the travelling time of the bottle 2 in the chamber 23. However, in the first aseptic chamber 23, the mist or gas of the sterilizing agent floating in the aseptic chamber 23 is condensed as high density hydrogen peroxide. Therefore, the virus entering the aseptic chamber 23 can be promptly and surely sterilized. Accordingly, the interiors of the aseptic chambers 23, 24, 26 and 27 can be maintained in the high aseptic state for a long time, enabling the packaging container excellent in high aseptic performance to be manufactured.

Within the first to fourth aseptic chambers 23, 24, 26 and 27, sterilizing apparatus (i.e., sterilizers) are disposed. The interiors of all the aseptic chambers 23, 24, 26 and 27 are sterilized by the sterilizers beforehand the starting of manufacturing the packaging containers. This sterilization is performed by spraying the sterilizing agent, jetting the heated water, releasing the water, or like, and in such sterilization, although the spores of the bacteria may still remain in the chambers, the vegetative cells of the bacteria, molds and yeasts will be killed or sterized.

### Second Embodiment 2

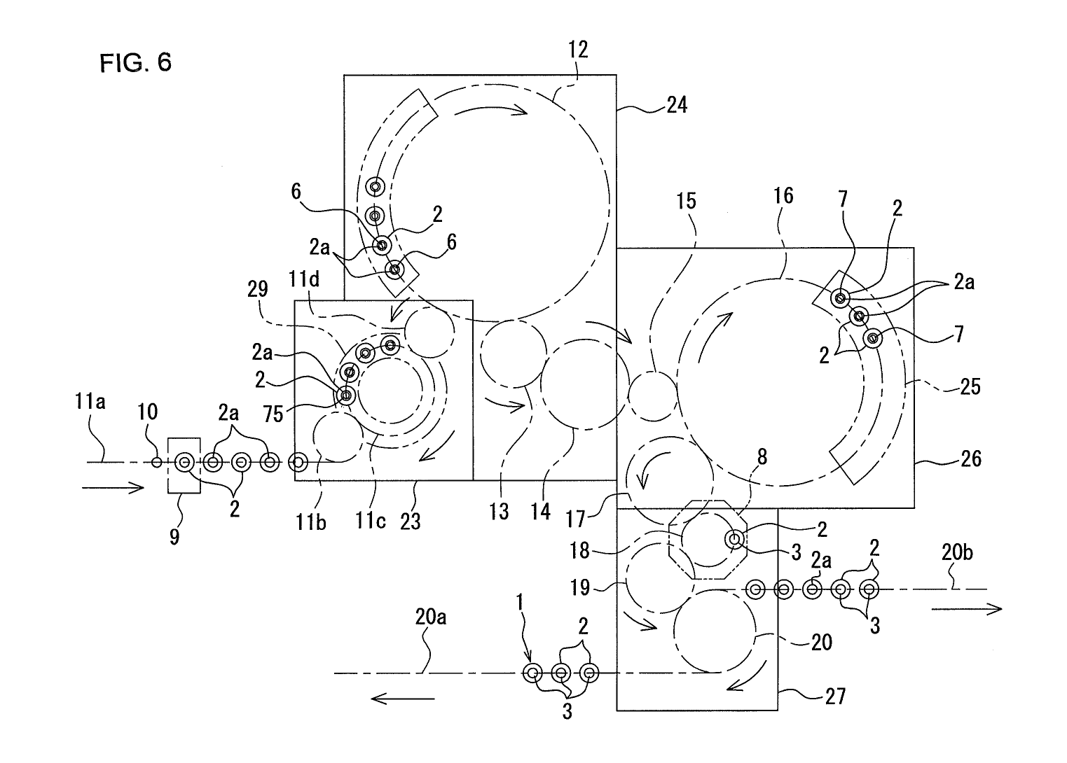

As shown in FIG. 6, in this embodiment 2, different from the first embodiment 1, wheels 11b and 11c are arranged between the introducing conveyer 11a and the wheel 12, and these wheels 11b and 11c are accommodated in the first aseptic chamber 23. The bottle 2 is conveyed from the introducing conveyer 11a to the wheel 11c through the wheel 11b, and sterilized by blowing the hydrogen peroxide gas “b” from a nozzle 75 while moving around the wheel 11c. Then, the bottle 2 reaches the wheel 12 through a wheel 11d and is sterilized by the heated water while moving around the wheel 12.

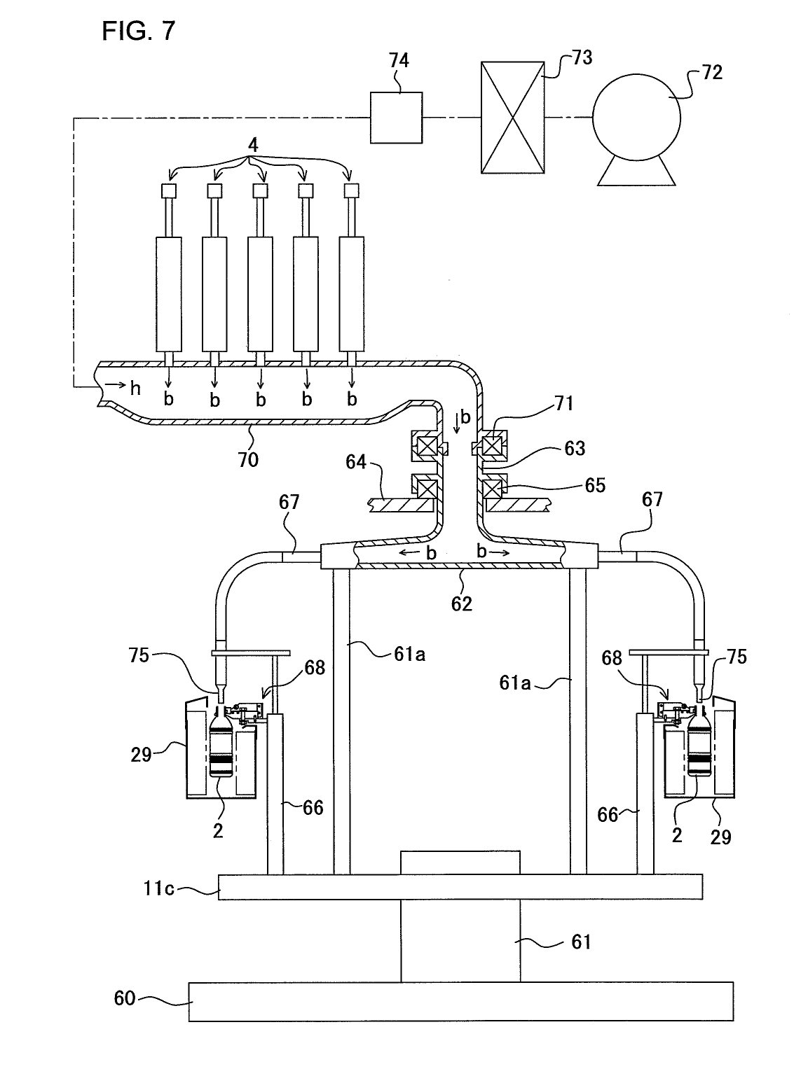

As shown in FIG. 7, the wheel 11c is mounted to a turning (swiveling) shaft 61 standing upward from a machine table 60 to be rotatable around the swiveling shaft 61. A support column 61a stands upward from a plate surface of the wheel 11c and a manifold 62 for jetting the hydrogen peroxide gas “b” is secured to the upper tip end portion of the column 61a. A conduit 63 extends upward on an extension line of the axis of the swiveling shaft 61 from an upper central portion of the manifold 62, and this conduit 63 is held to a support member 64, through a bearing 65, connected to the machine table 60. Accordingly, the manifold 62 becomes rotatable around the swiveling shaft 61 together with the wheel 11c.

Furthermore, another support column 66 also extends upward from the plate surface of the wheel 11c, and a holder 68 for holding the bottle 2 is mounted to an upper portion of the column 66. A plurality of such support columns 66 and holders 68 are arranged at predetermined pitches, respectively, around the wheel 11b. Since these holders 68 are coupled to the wheel 11c through the columns 66, the holders 68 are rotated in conformity with the rotating motion of the wheel 11c.

Hydrogen peroxide gas supply tubes 67 are disposed around the manifold 62 so as to extend toward the holders 68, respectively, and the nozzles 75 are mounted to front end portions of the respective support tubes 67. Each of the nozzles 75 is fixed to the support column 66, which has a front end to which an opening is formed. This opening directly faces the mouth portion 2a of the bottle 2 held by the holder 68. Accordingly, when the wheel 11c is rotated, the nozzles 75 are turned around the swiveling shaft 61 together with the bottles 2 held by the holders 68, and during this rotation, the hydrogen peroxide gas “b” is jetted into the bottles 2.

Further, tunnels 29 are disposed around the wheel 11b so as to surround passing ways of the bottles 2 held by the holders 68. The gas “b” discharged from the nozzle 75 flows outside the bottle 2, stays in the tunnel 29 as high density hydrogen peroxide mist, adheres to the outer surface of the bottle 2, or sterilizes the bacteria vegetative cells, molds, yeasts and the like floating in the tunnel 29.

A heating tube 70 is connected to the upper end portion of the conduit 63 of the manifold 62 through a seal member 71. The conduit 63 rotates integrally with the manifold 62 with respect to the heating tube 70, and the seal member 71 prevents the gas “b” from leaking through the connecting portion of the conduit 63 and the heating tube 70. To the heating tube 70, a plurality of hydrogen peroxide gas generators 4 are mounted so as to provide the hydrogen peroxide gas “b” to the heating tubes 70 from the hydrogen peroxide gas generators 4, respectively. The number of the hydrogen peroxide gas generators 4 to be operated may be determined in accordance with the amount of the gas “b” required for the sterilization of the bottles 2.

On the upstream side of the heating tubes 70, is disposed a hot air supplying device including a blower 72, a ULPA filter (Ultra Low Penetration Air Filter) 3, and an electric heater 74. The air sucked from the blower 72 is cleaned by the ULPA filter 73, heated by the electric heater 74 to a desired temperature, and fed to the heating tube 70 as hot air wind “h”. The hot air wind “h” is heated, for example, to more than 100° C. above the condensed point of the hydrogen peroxide, into aseptic air. This hot air wind “h” acts to carry the hydrogen peroxide gas “b” from the hydrogen peroxide gas generator 4 to the manifold 62, and is jetted into the bottle 2 from the nozzle 75 via the supply tube 67, or is flown outside the bottle 2.

When the hydrogen peroxide gas “b” is blown into the bottle 2 through the nozzle 75, the gas “b” changes into mist and adheres to the entire inner surface of the bottle 2, and the adhering mist is then condensed as the high density hydrogen peroxide which promptly sterilizes the inner surface of the bottle 2. The mist also adheres to the outer surface of the bottle 2 and is then condensed to thereby also promptly sterilize the outer surface thereof.

The bottle 2 of which inner and outer surfaces are sterilized is conveyed to the wheel 12 via the wheel 11d, and as like as the case of the first embodiment, the bottle 2 is thereafter sterilized by the heated water “c” injected from the nozzle 6.

In this embodiment 2, the mist “b” injected from the nozzle 75 fills the interior of the first aseptic chamber 23.

Within the first aseptic chamber 23, the virus adhering to the surface of the bottle 2 or entering together with air flow, which is caused by the travelling of the bottle 2, may invade into the aseptic chambers 23, 24, 26 and 27. However, the mist or gas of the sterilizing agent floating inside the aseptic chamber 23 changes to the hydrogen peroxide and is condensed as high density hydrogen peroxide water with respect to such virus. Thus, the virus entering the aseptic chamber 23 can be promptly and surely sterilized, and accordingly, the aseptic condition within the aseptic chambers 23, 24, 26 and 27 can be maintained with high aseptic performance for a long time, whereby packaging containers having high aseptic performance can be manufactured.

It is further to be noted that, with reference to this second embodiment, like reference numerals are added to members or portions corresponding to those of the first embodiment, and duplicated description is omitted herein.

### Third Embodiment 3

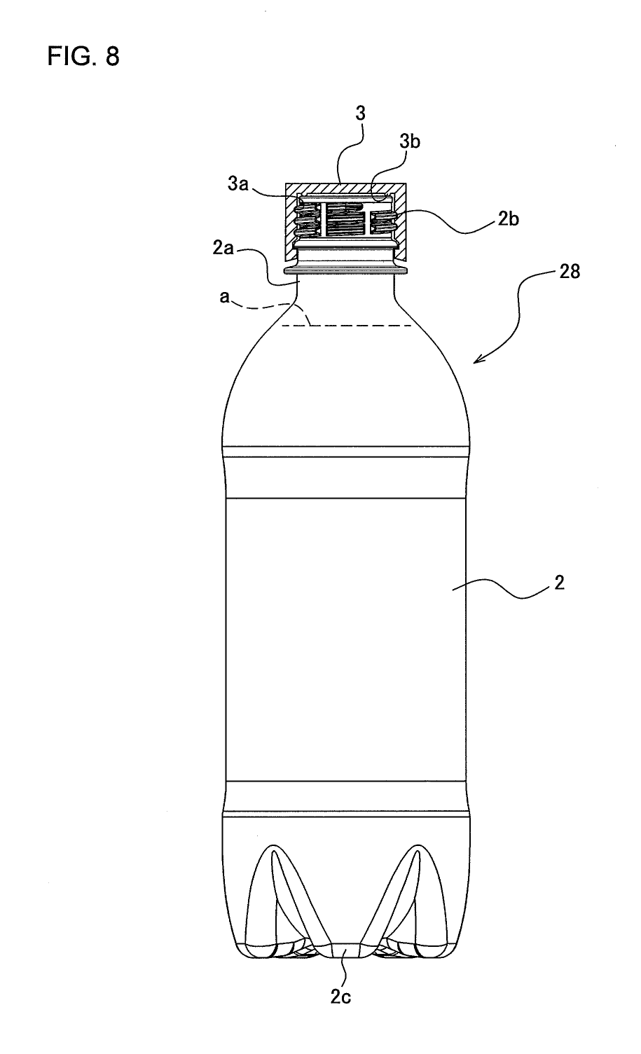

As shown in FIG. 8, a container of a packaging container 28 in this third embodiment 3 is formed as a pressure-resistant bottle having a petal shaped bottom-type or champagne-type bottom portion 2c. This bottle 2 is filled up with a beverage “a” having a nature for pressurizing the interior of the bottle with a gas, such as lactic carbonated drink, fruit-contained carbonated drink, colored-fruit carbonated drink or like.

As shown in FIG. 2, this beverage “a” is heated and sterilized (step 2), cooled (step 3), and thereafter, pressurized by applying carbonate gas (step 4). Then, the beverage fills the bottle 2 having a petal shaped bottom-type or champagne-type bottom portion 2c (step 5), and thereafter, substantially the same steps as those mentioned with reference to the first embodiment may be performed to manufacture the packaging container 28.

It is further to be noted that, with reference to this third embodiment, like reference numerals are added to members or portions corresponding to those of the first embodiment, and duplicated description is omitted herein.

### Example 1

Specimens (samples) A, B, C, D were prepared for inspecting the sterilization effects “(LRV (Long Reduction Value)=log(number of adhering molds/ln(total number/negative number)”, and results shown in Tables 1 and 2 were obtained.

TABLE 1
Sterilizing Test Result To Bottle Outer Surface

| | | | | | Cultivating | | | |
|---|---|---|---|---|---|---|---|---|
| | | | | | Result | | | |
| | | | | | to bottle | | | |
| | | | | | outer surface | | | |
| | H₂O₂ | Adhesion | Adhesion | | (positive/ | | | |
| | supply | amount to | amount to | | total BI | | | |
| | amount | inner surface | outer | Hot water | number) | | | Sterilizing |
| | (g/min) | (μL/bottle) | surface (μL/cm²) | rinsing (° C.) | 10³ | 10⁴ | 10⁵ | effect (LRV) |
| A | 20 | 6.4 | 0.074 | No | 0/5 | 0/5 | 5/5 | 5.1 |
| B | 40 | 12.1 | 0.142 | 70 | 0/5 | 0/5 | 0/5 | >6.1 |

With respect to the sterilization performance to the outer surface of the bottle, after the adhering of the hydrogen peroxide mist onto the outer surface by 0.142 μL/cm² from the “A” and “B” in the above Table 1, a rinsing process of the water heated to 70° C. with respect to the inner surface of the bottle was performed, and as a result, it was confirmed that the heat of the heated water is transferred to the outer surface of the bottle and the sterilization effect was improved to 6.1 LVR.

TABLE 2
Sterilizing Test Result To Bottle Inner Surface

| | | | | | Cultivating | | | |
|---|---|---|---|---|---|---|---|---|
| | | | | | Result | | | |
| | | | | | to bottle | | | |
| | | | | | inner surface | | | |
| | H₂O₂ | Adhesion | Adhesion | | (positive/ | | | |
| | supply | amount to | amount to | | total BI | | | |
| | amount | inner surface | outer | Hot water | number) | | | Sterilizing |
| | (g/min) | (μL/bottle) | surface (μL/cm²) | rinsing (° C.) | 10³ | 10⁴ | 10⁵ | effect (LRV) |
| C | no | 0 | 0 | 70 | 0/5 | 2/5 | 5/5 | 4.8 |
| D | 40 | 12.1 | 0.142 | 70 | 0/5 | 0/5 | 0/5 | >6.1 |

With respect to the sterilization performance to the inner surface of the bottle, the sterilization effect of 4.8 was obtained with respect to Chaeketomium as the ascomycete spore only by the rinsing of the heated water from “C” and “D” in the Table 2. However, by adhering the hydrogen peroxide mist by 12.1 μL to the inner surface of the bottle, it was confirmed that the sterilization effect of more than 6.1 LRV was obtained.

The sterilization effects to the outer surfaces of the bottles were obtained by using Aspergillus niger NBRC6341 as one kind of black molds as an index mold, and the sterilization effects to the inner surfaces of the bottles were obtained by using Chaetomium globosum NBRC6347 as one kind of ascomycete spores as an index mold. The sterilized BI for the outer surface and the mold-adhering bottle for the inner surface were cultivated for seven days at a temperature of 27° C. by using potato dextrose agar media and glucose sugar media, and thereafter, the remaining number of molds was calculated.

### Fourth Embodiment 4

Hereunder, the sterilization method and apparatus in a product line utilized in the first to third embodiments will be explained.

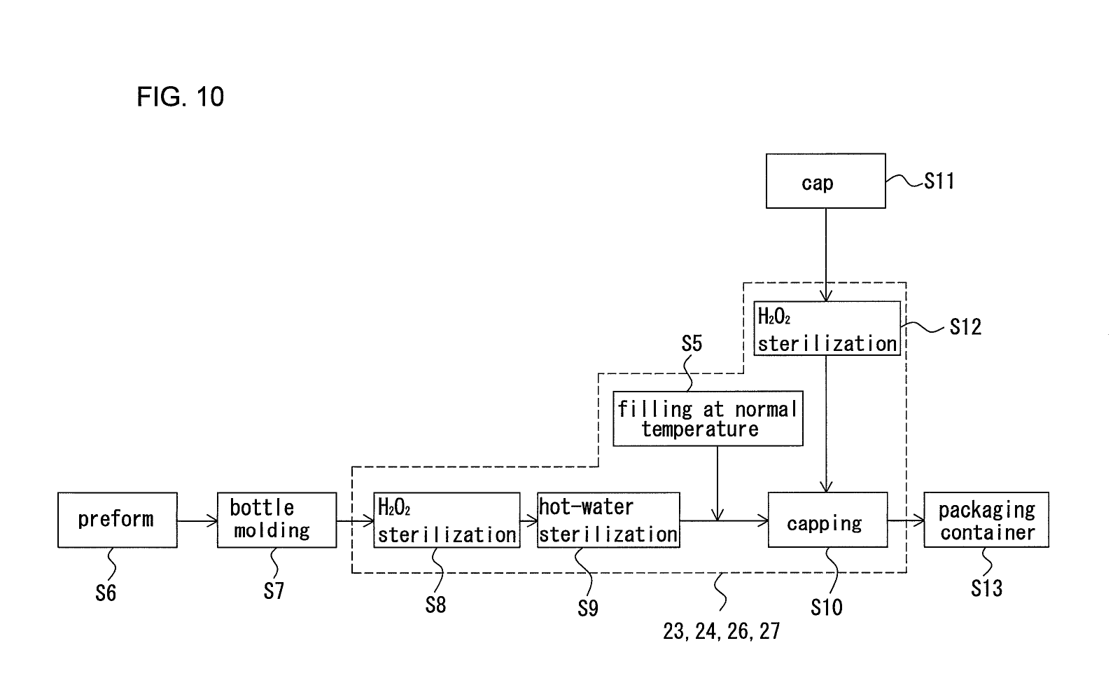

This product line is connected to a packaging line in an aseptic condition shown in FIG. 10.

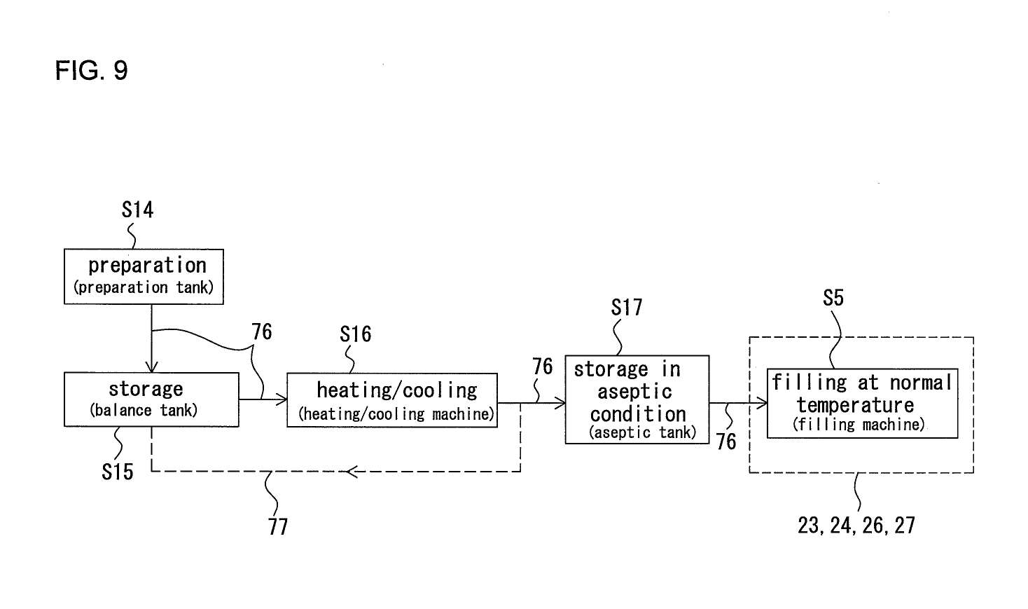

This aseptic packaging line is sterilized in a manner such that the spore of the bacteria can be allowed to be alive, but the vegetative cell, mold and yeast are not allowed to be alive. Moreover, the inner content such as beverage having acid level capable of suppressing the germination of the bacteria spore is fed from the product line shown in FIG. 9, and fills the PET (polyethylene telephthalate) bottle as a container.

The product line includes the steps of, as shown in FIG. 9, a beverage preparing step (S14), a storage step (S15) for storing the prepared beverage, a heating/cooling step (S16) for heating/cooling the prepared beverage, a storage step (S17) for storing the heated/cooled beverage in the aseptic condition, and a filling step (S5) at a normal temperature, which are performed in the described order, and this product line also includes a conduit 76 extending from a preparation tank for performing the beverage preparation and the filler (filling machine) for performing the filling step at the normal temperature.

For this conduit 76, a balance tank for the prepared beverage storing step (S15), a heating/cooling machine for the prepared beverage heating/cooling step (S16), and an aseptic tank for storing the heated/cooled beverage in the aseptic condition, are arranged in this order.

Further, the preparation tank, the balance tank, the heating/cooling machine and the filling machine are not shown in the drawings.

The preparation tank is a tank for preparing the beverage filling the container such as bottle, and the conduit 76 extends from this preparation tank to the succeeding balance tank.

The balance tank is a tank for storing the beverage fed from the preparation tank and acts as a buffer tank, which is disposed as occasion demands.

The sterilizing/cooling machine is specifically an ultra high temperature instantaneous sterilizer (UHT), and is capable of heating or cooling a fluid flowing inside the UHT sterilizer by switching the steps form heating to cooling (or vise versa). The UHT sterilizer can heat the fluid such as beverage of high temperature for a short time, and therefore, can minimally suppress damage to the beverage by the heat. Accordingly, it is possible to sterilize the beverage with flavor, taste, color and the like being maintained as they are. Further, the sterilizing/cooling machine and the balance tank are connected by means of a conduit 77 for returning the beverage from the sterilizing/cooling machine and the balance tank as shown with broken line.

The aseptic tank is a buffer tank disposed at a pre-stage of the filling machine (filler), so that, for example, when the filling machine temporarily stops, the beverage is stored.

The filling machine has, as shown in FIG. 3, the nozzle 7 so as to fill the bottle with a predetermined amount of beverage from the nozzle 7. The aseptic chamber 26 surrounds the filling machine as shown in FIG. 4 or 6, and the interior of the aseptic chamber 26 is held in the aseptic condition so as to fill the bottle with the beverage with the aseptic condition.

The beverage treated by the product line of the structures mentioned above is then processed as follows, and thereafter, is supplied to the packaging line.

The beverage is prepared in the preparation tank with a desired rate (S14). The thus prepared beverage is temporarily stored in the balance tank (S15), and thereafter, is heated to sterilize the beverage and then cooled by the heating/cooling machine (S16), thus performing necessary processes.

The heating temperature by this heating/cooling machine is about 90-98° C. at the acidity of pH 4.0, about 115-122° C. at the acidity of pH 4.0-4.6. According to this heating treatment, all the virus to be generated, in the beverage before the filling process, in the packaging container after the packaging, can be sterilized. That is, the beverage can be sterilized such that the bacteria spore can be allowed to be alive, but the vegetative cell, mold and yeast cannot be allowed to be alive. The bacteria spore remaining alive in the beverage is suppressed from germinating in the beverage having the acidity mentioned above.

Further, the cooling temperature of the beverage by the heating/cooling machine is a normal temperature of about 2-40° C. The beverage is cooled to a desired temperature by the heating/cooling machine.

The beverage cooled to the normal temperature is once stored in the aseptic tank (S17), and thereafter, is fed to the filling machine (S5).

The packaging line includes, as shown in FIG. 10, a preform supply step (S6), a bottle molding step (S7), a bottle sterilizing step (S8) by the hydrogen peroxide, a bottle sterilizing step (S9) by the heating water, a beverage filling step (S5) at a normal temperature, a capping step (S10), a cap supplying step (S11), a cap sterilizing step (S12), and a packaging container discharging step (S13), respectively.

Further, although the preform is not shown, the bottle, the cap and the packaging container are exemplarily shown in FIG. 1 or FIG. 8.

The bottle as a container is processed in the following manner by this packaging line, and thereafter, is formed as packaging container.

First, the preform is prepared (S6), and a bottle is molded through blow-molding process from the preform by using a blow molding machine, not shown, (S7). The bottle may be molded from a resin material such as polypropylene, polyethylene or the like other than the PET.

The sterilizing process is performed to the inner and outer surfaces of the bottle by the hydrogen peroxide and the heated water (S8 and S9). The processes from the preform supply step (S6) to the sterilizing step (S8) through the bottle molding step (S7) may be separately in place or time, but it is desirable to perform such processes continuously.

The hydrogen peroxide is converted into gas or mist by using a hydrogen gas generating device, and the gas or mist is injected from the nozzle toward the bottle. The hydrogen peroxide adheres to the entire inner surface of the bottle to thereby sterilize the vegetative cells, molds and yeasts in the bottle. The sterilizing force of this hydrogen peroxide is determined to an extent such that the bacteria vegetative cells, molds and yeasts can be sterilized, but the bacteria spores cannot be sterilized. Accordingly, the amount of the hydrogen peroxide to be used can be reduced.

Furthermore, the mist or gas of the hydrogen peroxide can sterilize the bacteria vegetative cells, molds and yeasts adhering to the outer surface of the bottle. Thus, since the outer surface of the bottle is also sterilized, the inversion of the bacteria vegetative cells, molds and yeasts adhering to the outer surface of the bottle into the bottle can be prevented, and hence, the beverage filling the bottle can be prevented from being contaminated thereby.

The bottle of which inner and outer surfaces are sterilized is then sterilized by the heated water (S9). More specifically, the water heated to about 65 to 75° C. is supplied from the nozzle into the bottle. The heated water entering the bottle circulates in the bottle and then flows outward externally of the bottle. By supplying this heated water, mold having anti-medical agent property as a part of ascomycete and the like damaged by the hydrogen peroxide can be sterilized too. Furthermore, an excessive hydrogen peroxide remaining inside the bottle can be also washed out of the bottle by the heated water.

The beverage sterilized and then cooled to the normal temperature then fills the bottle sterilized by the heated water (S5). The temperature of the beverage at the filling time is about 2 to 40° C.

Specifically, the filling of the beverage is performed, as shown in FIG. 3, by placing the nozzle so as to face the mouth portion of the bottle and feeding the beverage into the bottle. As mentioned hereinbefore, it is desired for the acidity of the beverage to be less than pH 4.6, and more preferably, less than pH 4.0, the beverage being such as tomato juice, vegetable juice, functional drink, carbonated lemon juice, grape juice, fruit juice or the like.

The bottle filled up with the beverage by a predetermined amount is sealed by the cap (S10). A number of caps are preliminarily collected (S11), and arranged in a row directing toward the bottles filled up with the beverage. The hydrogen peroxide gas or mist is blasted against the inner and outer surfaces of the caps to be thereby sterilized (S12), and then, the caps are contacted to the mouth portions of the bottles and screwed or fastened by means of screws or like.

The bottle sealed by the cap is discharged out of the packaging line as a product packaging container (S13).

During the operations in the product line and the packaging line, at least the respective steps of the beverage filling step at the normal temperature (S5), the bottle sterilizing step by the hydrogen peroxide (S8), the bottle sterilizing step by the heated water (S9), the capping step (S10) and the cap sterilizing step (S11) are performed under the aseptic environment surrounded by the aseptic chambers 23, 24, 26 and 27, respectively.

It is necessary for the product line and the packaging line mentioned above to be subjected to the sterilization treatment beforehand the manufacturing of the packaging container so as not to mix and breed the virus inside the packaging container.

This sterilization treatment or process will be performed by a manner described hereunder.

With the packaging line, the interiors of the aseptic chambers 23, 24, 26 and 27 shown in FIG. 4 or 6 are subjected to the sterilizing process. This sterilizing process is performed to the extent such that the sterilizing effects to the interiors of these chambers substantially identical to those to the beverage and the bottle are obtainable by spraying the hydrogen peroxide and, thereafter, the heated water into the aseptic chambers 23, 24, 26 and 27. According to such treatment, in the aseptic chambers 23, 24, 26 and 27, the bacteria spore can remain alive, but the bacteria vegetative cells, molds and yeasts are sterilized.

After the above sterilizing process, positive aseptic air is always fed into the aseptic chambers 23, 24, 26 and 27 so that aseptic air is blown out externally from the aseptic chambers 23, 24, 26 and 27 from the interiors thereof.

With the product line, the following sterilizing process will be performed.

First, the heated water of a predetermined temperature is fed to the product line. The temperature of the heated water is for example 85° C. and is fed for 30 min, for example.

This heated water flows inside the preparation tank, the balance tank, the heating/cooling machine, the aseptic tank, and the filler corresponding to the respective steps of (S14) to (S5) shown in FIG. 9 so as to heat and sterilize the interiors of these equipments and the conduits 76 and 77. The heated water is produced by the heating/cooling machine as heating sterilizing means, circulates between the balance tank and the heating/cooling machine through the returning conduit 77, and is fed to the preparation tank, the aseptic tank and the filler.

According to the heating treatment by the heated water, in the product line, as like as the sterilization effects to the beverage and the bottle, the bacteria spore remains alive, but the bacteria vegetative cells, molds and yeasts are sterilized.

After the treatment by the heated water, aseptic air like the positive aseptic air fed inside the aseptic chambers 23, 24, 26 and 27 is always fed into the product line to thereby keep the inside of the product line with the positive pressure.

Further, it may be possible to use released steam or pressurized steam in place of the heated water mentioned above. The released steam is a steam which is fed without being pressurized under the atmospheric pressure.

Thereafter, aseptic water of the normal temperature or less than normal temperature is fed to the product line so as to flow therein, thereby cooling the interior of the product line to the normal temperature of about 2 to 40° C., which is however set in accordance with the nature of the beverage.

Substantially identical cooling effect will be obtainable by flowing the beverage of the normal temperature or less than normal temperature in place of such aseptic water, and in such case, the sterilizing step in the product line can be smoothly switched to the filling step.

The aseptic water is obtained from the heated water by cooling the same by switching the operation of the heating/cooling machine to the cooling use. The interior of the product line can be cooled to the normal temperature by flowing this cooled water in the product line as like as the flowing of the heated water.

According to the above process, the product line for filling the packaging container such as bottle with an acidic beverage having relatively high acidity, allowing the spore to remain, but not being suitable for the hot-pack method, can be easily sterilized for a short time, and therefore, the packaging line can promptly start to operate, thus enhancing the production efficiency of the packaging containers.

### Fifth Embodiment 5

The environment in which the aseptic filling process was performed, in the embodiments 1 to 4, is maintained in an aseptic condition by an aseptic environment maintaining means.

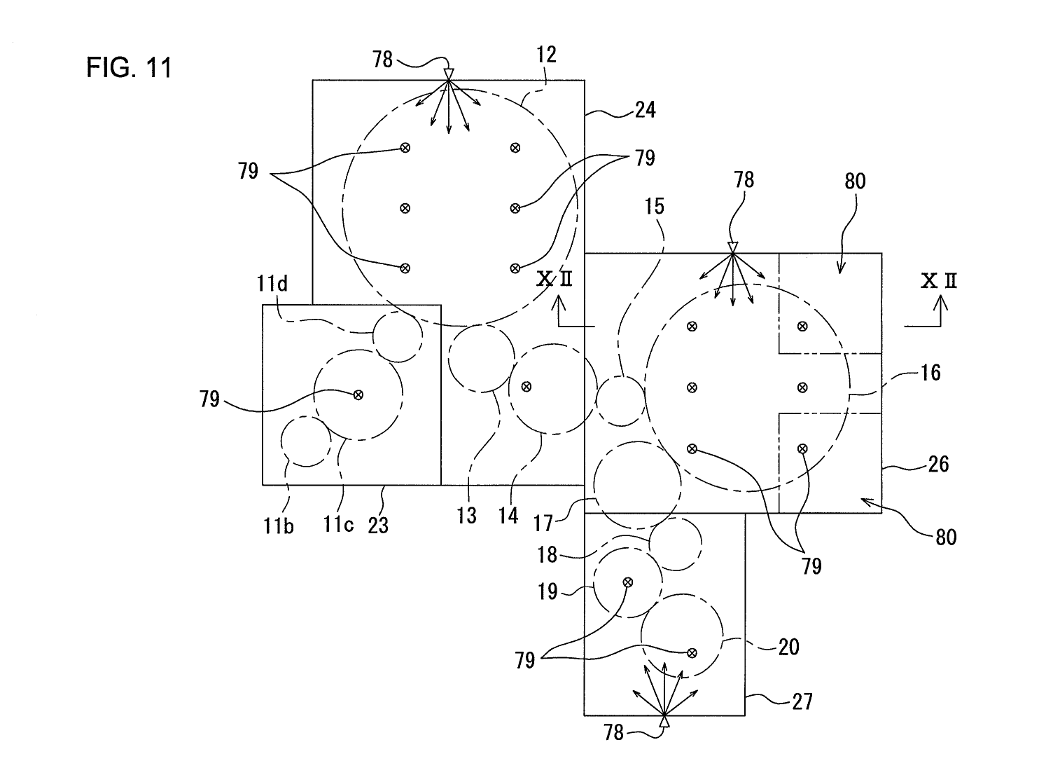

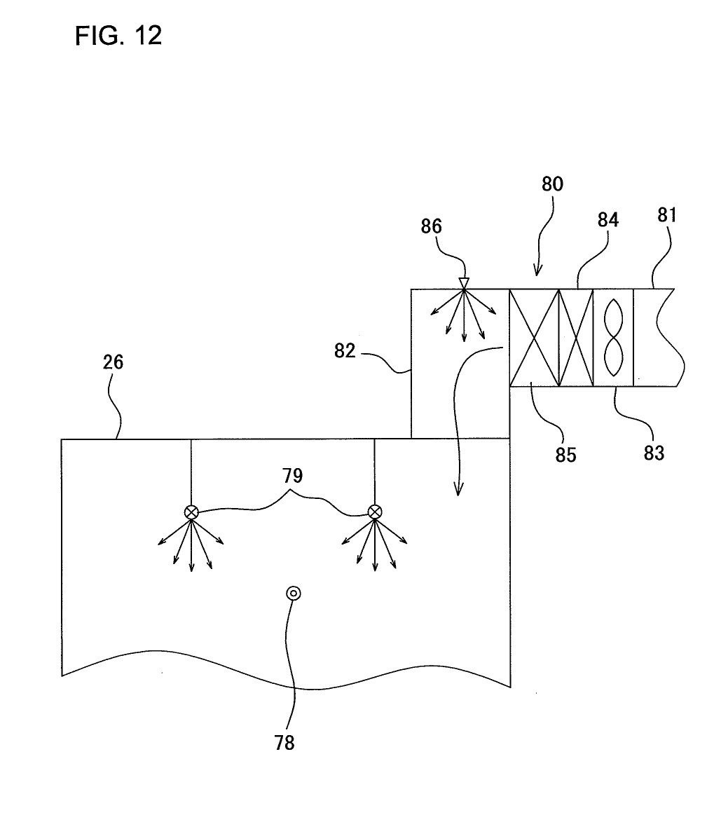

That is, as shown in FIG. 4 or FIG. 6, a first sterilization treatment means, a second sterilization treatment means, an inner content filling means, a sealing means and the like are covered by the aseptic chambers 23, 24, 26 and 27 so as to be shut off from an outer environment. As shown in FIGS. 11 and 12, a sterilizing agent spray 78, a heated water spray nozzle 79 and an aseptic air supply device 80 are disposed in each of the aseptic chambers 23, 24, 26 and 27.

The sterilizing agent sprays 78 are disposed so that the sterilizing agent adheres to the entire area within the respective aseptic chambers 23, 24, 26 and 27. Hydrogen peroxide is used as such sterilizing agent, and two-fluid nozzles utilizing compressed air for the spraying of the hydrogen peroxide are utilized as the sterilizing agent spray nozzles 78.

The hydrogen peroxide sprayed from the sterilizing agent spray nozzles 78 adheres to the entire areas in the respective aseptic chambers 23, 24, 26 and 27.

In place of the hydrogen peroxide, acetyl hydrogen peroxide may be used. Further, it may be possible to spray the hydrogen peroxide to perform the sterilizing treatment after the spraying the acetyl hydrogen peroxide to perform the sterilizing treatment.

The heated water spray nozzles 79 are arranged so that the heated water is sprayed entirely in the respective aseptic chambers 23, 24, 26 and 27. The heated water may be supplied from the heated water supply source used for the sterilization of the bottle in the afore-mentioned embodiments, and the water heated to about 80 to 100° C. is sprayed into the aseptic chambers 23, 24, 26 and 27, respectively. Spray nozzles using, for example, spin balls may be used as the heated water spray nozzles.

The heated water sprayed from the heated water spray nozzles 79 adheres to the entire areas in the respective aseptic chambers 23, 24, 26 and 27.

Two units of aseptic air supply devices 80 are prepared, and ducts or conduits are connected from these devices 80 to a ceiling of the aseptic chamber 26. Each of these ducts, as shown in FIG. 12, is provided with a horizontal portion 81 and a suspended portion 82 suspended downward from the horizontal portion 81 toward the ceiling of the aseptic chamber 26. Inside the horizontal portion 81, are arranged a blower 83, a heater 84, a ULPA Filter (Ultra Low Penetration Air Filter) 85 in the described order from the upstream side to the downstream side.

When the blower 83 is rotated, ambient (external) air is brought into the ducts, and this ambient air is heated by a heater 84 to a temperature of about 100° C. into hot air, which is then subjected to dust exhaustion and sterilization filtering into aseptic air, which is then flown into the aseptic chamber 26. This aseptic air flows from the aseptic chamber 26 into the other aseptic chambers 23, 24 and 27 and stays in all these chambers to create positive pressure atmosphere. Thereafter, the positive aseptic air flows outside through the inlet/outlet (entrance/exit) portion of the bottle 2 in each of the aseptic chambers 23 and 27. Thus, an ambient air including dust, bacteria and like can be prevented from entering inside the aseptic chambers 23, 24, 26 and 27.

A sterilizing agent spray nozzle 86 is attached to a perpendicular portion 82 of the duct mentioned above. Beforehand the manufacturing of the packaging container 1, the surface of the ULPA filter 85 and the interior of the perpendicular portion 82 are sterilized by the hydrogen peroxide sprayed from the sterilizing agent spray nozzle 86.

Hereunder, means for keeping the aseptic environment condition mentioned above will function as follows.

The interiors of the aseptic chambers 23, 24, 26 and 27 have been subjected to the sterilization treatment beforehand the aseptic air filling process.

The hydrogen peroxide is sprayed through the respective sterilizing agent spray nozzles 78 and 86, and the atomized hydrogen peroxide adheres to the entire area inside the respective aseptic chambers 23, 24, 26 and 27. According to the spraying of the hydrogen peroxide, the bacteria vegetative cells, molds and yeasts in the respective aseptic chambers 23, 24, 26 and 27 can be sterilized, and in addition, the interior of the perpendicular portion 82 of the duct and the surface of the ULPA filter 85 can be also sterilized.

After the completion of the spraying of the hydrogen peroxide, the heated aseptic air is fed into the aseptic chambers 23, 24, 26 and 27 by the actuation of the blower 83 of the aseptic air supply device 80. By this heated aseptic air, the hydrogen peroxide adhering to the interior of the respective aseptic chambers 23, 24, 26 and 27 can be dried and then removed.

Thereafter, the heated water is sprayed from the heated water spray nozzle 79 to the entire air inside the aseptic chambers 23, 24, 26 and 27, respectively, thereby sterilizing partially the ascomycete damaged by the hydrogen peroxide.

Since the duct of the aseptic air supply device 80 is provided with the perpendicular portion 82, the sprayed heated water is blocked from adhering to the ULPA filter 85 by the location of this perpendicular portion 82.

The hydrogen peroxide of the predetermined density is supplied at the predetermined rate for the predetermined time, and the heated water of the predetermined temperature is also supplied at the predetermined rate for the predetermined time.

Thus, the interiors of the respective aseptic chambers 23, 24, 26 and 27 become the aseptic condition or state to the extent in which the bacteria spore can be allowed to be alive, but the bacteria vegetative cells, molds and yeasts cannot be allowed to be alive.

This aseptic condition can be maintained by always supplying the aseptic air into the aseptic chambers 23, 24, 26 and 27 by the aseptic air supply device 80.

As mentioned above, after the sterilization of the interiors of the aseptic chambers 23, 24, 26 and 27, the first sterilizing means, the second sterilizing means, the inner content filling means and the sealing means are operated to thereby start the manufacturing of the aseptic packaging container.

Further, the present invention is not limited to the described embodiments 1-5, and many other changes and modifications may be made within the scope of the present invention. For example, in the above embodiments 1-5, although the PET bottles are processed as containers to be sterilized, the present invention is applicable, other than the PET bottles, to bottles such as made of polypropylene material, evaporated PET material, polyethylene material, glass or like material, and also applicable, other than bottles, to cup-shaped container or like.

Furthermore, although in the above embodiments 1-5, the hydrogen peroxide is utilized as the sterilizing agent, acetyl hydrogen peroxide (peracetic acid) group sterilizing agent may be also applied in place of the hydrogen peroxide, and in such case, water of normal temperature may be used in place of the heated water.

1. A packaging container wherein an interior of a container is sterilized by a sterilizing agent and a heated water so as to allow bacterial spore to be alive but not to allow bacteria vegetative cell, mold and yeast to be alive, an inner content, which has been subjected to a sterilization treatment, having an acidity suppressing germination of the bacteria spore fills the container at a normal or low temperature, and the container is sealed by a lid.

2. The packaging container according to claim 1, wherein the sterilizing agent is hydrogen peroxide, and the interior of the container is sterilized by blowing mist or gas of the hydrogen peroxide into the container.

3. The packaging container according to claim 2, wherein the mist of the hydrogen peroxide to be fed into the container is of an amount of 5 to 50 μL/container.

4. The packaging container according to claim 2, wherein the gas of the hydrogen peroxide to be fed into the container has a gas density of 1 to 5 mg/L.

5. The packaging container according to claim 1, wherein the heated water has a temperature of 65 to 85° C. and is fed at a feeding rate of 5 to 15 L/min.

6. The packaging container according to claim 1, wherein the inner content has an acidity of less than pH 4.6.

7. The packaging container according to claim 1, wherein the inner content fills the container at a normal temperature of 3 to 40° C.

8. The packaging container according to claim 1, wherein the inner content is a liquid beverage.

9. The packaging container according to claim 1, wherein the container is made of PET (polyethylene telephthalate) material or polyethylene material.

10. The packaging container according to claim 1, wherein the container is a bottle.

11. A method of manufacturing a packaging container, comprising the steps of: sterilizing an interior of a container by a sterilizing agent and a heated water so as to allow bacterial spore to be alive but not to allow bacteria vegetative cell, mold and yeast to be alive; filling the container with an inner content, which has been subjected to a sterilization treatment, having an acidity suppressing germination of the bacteria spore at a normal or low temperature; and sealing the container by a lid.

12. The method of manufacturing a packaging container according to claim 11, wherein the interior of the container is sterilized by blowing mist or gas of a hydrogen peroxide as sterilizing agent into the container.

13. The method of manufacturing a packaging container according to claim 11, wherein the mist of the hydrogen peroxide to be fed into the container is of an amount of 5 to 50 μL/container.

14. The method of manufacturing a packaging container according to claim 11, wherein the gas of the hydrogen peroxide to be fed into the container has a gas density of 1 to 5 mg/L.

15. The method of manufacturing a packaging container according to claim 11, wherein the heated water has a temperature of 65 to 85° C. and is fed at a feeding rate of 5 to 15 L/min.

16. The method of manufacturing a packaging container according to claim 11, wherein the inner content has an acidity of less than pH 4.6.

17. The method of manufacturing a packaging container according to claim 11, wherein the inner content fills the container at a normal temperature of 3 to 40° C.

18. The method of manufacturing a packaging container according to claim 11, wherein the inner content is a liquid beverage.

19. The method of manufacturing a packaging container according to claim 11, wherein the container is made of PET (polyethylene telephthalate) material or polyethylene material.

20. The method of manufacturing a packaging container according to claim 11, wherein the container is a bottle.

21. The method of manufacturing a packaging container according to claim 20, wherein the bottle is subjected to a blow-molding process from a preform just before the sterilization treatment of the interior of the bottle.

22. The method of manufacturing a packaging container according to claim 11, wherein an exterior of the container is sterilized by the sterilizing agent and the interior of the container is sterilized by the heated water in a state in which the sterilizing agent adheres to an outer surface of the container.

23. An apparatus for manufacturing a packaging container comprising: a conveying unit that conveys a container along a predetermined conveying root; a first sterilizing unit that is disposed along the conveying root for performing the sterilization of an interior of the container by a sterilizing agent so as to allow bacterial spore to be alive but not to allow bacteria vegetative cell, mold and yeast to be alive; a second sterilizing unit that performs the sterilization of an interior of the container by a heated water so as to allow bacterial spore to be alive but not to allow bacteria vegetative cell, mold and yeast to be alive; a content filling unit that fills the container with an inner content, which has been subjected to a sterilization treatment, having an acidity suppressing germination of the bacteria spore at a normal or low temperature; and a sealing unit that seals the container by a lid, wherein the respective units are arranged in the described order, and a portion between the first sterilizing unit to the sealing unit is covered by an aseptic chamber.

24. The apparatus for manufacturing a packaging container according to claim 23, wherein the sterilizing unit is composed of a nozzle for blowing mist or gas of hydrogen peroxide as the sterilizing agent into the container, and the nozzle has a front end facing a mouth portion of the container.

25. The apparatus for manufacturing a packaging container according to claim 23, wherein the container is a bottle and a blow-molding unit that molds the bottle from a preform is disposed just before the sterilizing unit.

26. The method of manufacturing a packaging container according to claim 11, wherein with respect to a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive, the interior of the product line is positively pressurized, thereafter, the interior of the product line is cooled to a normal temperature by passing an aseptic water or the content, and the content is fed to the container through the thus cooled product line.

27. The method of manufacturing a packaging container according to claim 26, wherein the heated water has a temperature of 80 to 140° C. and passes through the product line for 1 to 30 minutes.

28. The apparatus for manufacturing a packaging container according to claim 23, further comprising: a heating and sterilizing unit that sterilizes and positively pressurizes an interior of a product line in which a content, which is sterilized so as to allow bacterial spore to be alive but not to allow bacteria vegetative cell, mold and yeast to be alive, and which has an acidity suppressing germination of the bacteria spore, flows from a preparation tank of the content to a nozzle of a filling machine, a heated water of a predetermined temperature, a released steam, or a pressurized steam passes for a predetermined time, an interior of the product line is sterilized so as to allow bacterial spore to be alive but not to allow bacteria vegetative cell, mold and yeast to be alive; and a cooling unit that cools the interior of the product line to a normal temperature by passing an aseptic water or content through the product line after the sterilization by the heating and sterilizing unit.

29. The apparatus for manufacturing a packaging container according to claim 28, wherein the heated water has a temperature of 80 to 140° C. and passes through the product line for 1 to 30 minutes.

30. The packaging container according to claim 28, wherein the inner content has an acidity of less than pH 4.6.

31. The method of manufacturing a packaging container according to claim 11, wherein steps of sterilizing the interior of the container, filling the container with the content, and sealing the container with the lid are performed inside the aseptic chamber, the interior of the aseptic chamber being preliminarily sterilized by the sterilizing agent and the heated water so as to allow bacterial spore to be alive but not to allow bacteria vegetative cell, mold and yeast to be alive.

32. The apparatus for manufacturing a packaging container according to claim 23, wherein the first sterilizing unit, the second sterilizing unit, the content filling unit and the sealing unit are covered by the aseptic chamber, in which a nozzle is disposed so as to inject the sterilizing agent and the heated water successively, and by successively injecting the sterilizing agent and the heated water through the nozzle, the interior of the aseptic chamber is preliminarily sterilized so as to allow bacterial spore to be alive but not to allow bacteria vegetative cell, mold and yeast to be alive.
